(19) United States
(12) Patent Application Publication
(10) Pub. No.: US 2023/0210901 A1
(43) Pub. Date: Jul. 6, 2023

(54) OVERCOMING THE TUMOR MICROENVIRONMENT FOR CELL THERAPY BY TARGETING MYELOID DERIVED SUPPRESSOR CELLS THROUGH A TRAIL-R2 SPECIFIC RECEPTOR

(71) Applicant: Baylor College of Medicine, Houston, TX (US)

(72) Inventors: Valentina Hoyos, Houston, TX (US); Saisha Nalawade, Houston, TX (US); Ann Marie Leen, Houston, TX (US); Helen E. Heslop, Houston, TX (US); Juan Fernando Vera Valdes, Houston, TX (US); Malcolm Brenner, Houston, TX (US); Cliona M. Rooney, Bellaire, TX (US)

(21) Appl. No.: 17/996,759

(22) PCT Filed: Apr. 21, 2021
(86) PCT No.: PCT/US2021/028437

§ 371 Date: Oct. 20, 2022

Related U.S. Application Data
(60) Provisional application No. 63/014,382, filed on Apr. 23, 2020.

(51) Int. Cl. A61K 35/17; C07K 16/28; C07K 14/725; C07K 14/705; A61K 39/395; A61K 38/17; A61P 35/00

(52) U.S. Cl. CPC A61K 35/17; C07K 16/2878; C07K 14/7051; C07K 14/70578; A61K 39/3955; A61K 38/177; A61K 38/1774; A61P 35/00; C07K 2317/622; C07K 2319/33; C07K 2319/03; A61K 2039/5156

(57) ABSTRACT

Embodiments of the disclosure include methods and compositions for inhibiting the immune suppressive tumor microenvironment using cell therapy wherein the cells express a chimeric protein having an extracellular domain that binds TRAIL-R2 and an intracellular domain that in specific embodiments comprises one or more costimulatory domains that enhance activity of the cells upon activation. In specific embodiments, the chimeric protein comprises an scFv that targets TRAIL-R2 and an intracellular region that comprises a costimulatory domain from 4-1BB. In particular embodiments, the cells also express a therapeutic protein, such as a chimeric antigen receptor.

### CROSS-REFERENCE TO RELATED APPLICATIONS

This application claims priority to U.S. Provisional Patent Application Ser. No. 63/014,382, filed Apr. 23, 2020, which is incorporated by reference herein in its entirety.

### TECHNICAL FIELD

Embodiments of the disclosure include at least the fields of cell biology, molecular biology, immunology, and medicine including at least cancer medicine.

### BACKGROUND

Breast cancer (BC) is the most common malignancy in women worldwide and a leading cause of cancer related deaths¹. Triple negative breast cancer (TNBC) is a heterogeneous group of tumors and accounts for 15-20% of all BC cases^{2,3}. Patients with TNBC have limited treatment options due to lack of effective targeted therapies otherwise available for other BC subtypes. Thus, new targets and effective therapeutic strategies are urgently needed for the treatment of TNBC. CAR T cell immunotherapies have demonstrated remarkable success in the treatment of hematological malignancies^{4,5}. However, CAR T cell treatment of solid tumors like BCs has proven more challenging, for the most part due to the hostile tumor microenvironment (TME).

The TME of some solid tumors, including BC, is characterized by presence of chemokines like CXCL5, CCL2, CCL5 which lead to recruitment of myeloid cells from the bone marrow into the tumor tissue and suppressive cytokines such as G-CSF, GM-CSF and IL-6, which result in the differentiation of these cells into myeloid derived suppressor cells (MDSCs)⁶⁻¹². Increased level of MDSCs in the circulation and at tumor site is correlated with advanced clinical stage and increased metastatic disease burden^{13,14}. Single cell transcriptomics has revealed a MDSC specific gene signature¹⁵. MDSCs have been shown to promote tumor growth, metastasis and immune suppression and their presence in tumors and blood of patients has been associated with poor response to immunotherapies in patients¹⁶. MDSCs inhibit effector T cell functions through several mechanisms including (1) promoting T regulatory cells (Tregs) (2) production of reactive oxygen species (ROS) (3) secretion of anti-inflammatory cytokines including IL-10 and TGFβ (4) depletion of important amino acids (arginine and tryptophan) necessary for T cell proliferation (5) induction of arginase (Arg) and indoleamine 2, 3-dioxygenase (IDO) and (6) expression of PD-L1. Additionally, they actively shape the TME through cross-talk with BC cells and the surrounding stroma, leading to increased angiogenesis, tumor invasion and metastasis by remodeling the extracellular matrix via production of MMP9^{15,17}.

Armed with these facts, investigators have focused on finding ways to eliminate MDSCs from the TME. Most strategies are non-specific and have significant side effects. However, a targeted approach is to use an agonist mAb against TNF-related apoptosis induced ligand-receptor 2 (TR2); a receptor expressed on MDSCs that activates apoptosis upon engagement with its soluble ligand, TRAIL¹⁸. A clinical trial using a TR2 agonistic antibody (DS-8273a) demonstrated that treatment significantly reduced MDSC numbers in the peripheral blood and at the tumor site and this was associated with longer progression free survival¹⁹.

In order to target solid tumors, CAR T cells targeting Mucin 1 (MUC1) were previously generated and validated²⁰. MUC1 is a glycoprotein that is heavily glycosylated on normal healthy cells and hypoglycosylated with a predominance of shorter core 1 glycans on cancer cells, enabling tumor selective targeting of the hypoglycosylated form, thereby reducing issues related to ‘on target off tumor toxicity’²¹⁻²³.

The present disclosure provides solution of long-felt needs in the art of enhancing cellular therapy.

The present disclosure provides solution of long-felt needs in the art of enhancing cellular therapy.

### BRIEF SUMMARY

Embodiments of the disclosure concern methods and compositions related to cellular therapy and improvements over known cellular therapies in the art. The cellular therapy of the disclosure is enhanced in activity over other cellular therapies that are not produced as described herein. In particular embodiments, the cellular therapy is modified so that it has improved activity upon use in vivo, including improved activity with any type of cancers, including those that have cells that express TRAIL-R2, such as MDCSs, and including solid tumors and hematological cancers. In specific embodiments, the cellular therapy provides for improved activity in the TME to allow for enhanced killing of tumor cells therein. In specific cases, the cellular therapy comprises a chimeric protein that provides activity to the cells for overcoming immune suppression of any kind in the TME, including at least overcoming inhibition of activity by MDSCs. The cells may or may not also comprise a CAR, transgenic TCR or native TCR, or killer-cell immunoglobulin-like receptors (KIR), for example. Therapeutic cells expressing the chimeric protein allow those cells to induce apoptosis in target cells in the TME, including MDSCs.

In particular embodiments, the cellular therapy has improved activity by being able to target the TRAIL-R2 protein. In specific aspects, a chimeric protein that binds TRAIL-R2 through an extracellular domain is able to inhibit target cells that express TRAIL-R2, including cells in the TME such as MDSCs in addition to macrophages, polynuclear monocytes, T regulatory cells, fibroblasts, endothelial cells, or a combination thereof. The TRAIL-R2-targeting chimeric protein is delivered to the TME on cells that themselves, without further modification, may be therapeutic against cancer, or the cells have additional modification(s) that are therapeutic against cancer or that render the cells as therapeutic against cancer. In specific embodiments, the chimeric protein comprises one or more agents that bind TRAIL-R2 and also comprise an intracellular region. In specific cases, the intracellular region may be of any kind and may include one or more domains, such as one or more domains that enhance activity of the cells expressing the chimeric protein. In specific cases, the one or more domains enhance expansion, persistence, and/or activity of the cells. In such cases, the cells may be stem cells of any kind (including at least mesenchymal stem cells or induced pluripotent stem cells) or immune cells of any kind, including at least T cells, NK cells, NK T cells, etc. In certain embodiments the intracellular region comprises one or more costimulatory domains such that upon binding to a target, the cells expressing the chimeric protein may be co-stimulated to have enhanced persistence and expression.

Cells of any kind may express the chimeric protein and also express another heterologous protein, including a therapeutic protein such as a chimeric antigen receptor (CAR). The chimeric antigen receptor may target any one or more antigens, including any one or more cancer antigens. In specific embodiments, the chimeric antigen receptor (in addition to the TRAIL-R2-targeting chimeric protein) comprises one or more costimulatory domains that upon binding to its target antigen the cells are co-stimulated to have enhanced persistence and expression. In some cases, the enhanced persistence and expression from co-stimulation by the TRAIL-R2-targeting chimeric protein is incremental with respect to the enhanced persistence and expression from co-stimulation from the CAR, in some cases the enhanced persistence and expression from co-stimulation by the TRAIL-R2-targeting chimeric protein is additive with respect to the enhanced persistence and expression from co-stimulation from the CAR, although in alternative cases the enhanced persistence and expression is synergistic between the co-stimulation from the TRAIL-R2-targeting chimeric protein and the co-stimulation from the CAR.

To potentiate expansion and persistence of MUC1 CAR T cells and modulate the suppressive TME, provided herein is one example of a novel chimeric co-stimulatory receptor, TRAIL-R2.4-1BB, encoding a scFv derived from the DS-8273a agonistic antibody followed by a 4-1BB endodomain. In particular embodiments, engagement with TRAIL-R2 expressed on TME-resident MDSCs leads to both MDSC apoptosis and CAR T cell co-stimulation, promoting persistence and expansion of CAR T cells at the tumor site and improved tumor killing.

Embodiments of the disclosure include polynucleotides comprising an expression construct that encodes a chimeric protein, wherein said chimeric protein comprises (1) an extracellular region comprising a TNF-related apoptosis-inducing ligand Receptor 2 (TRAIL-R2)-binding agent; and (2) an intracellular region that is not from TRAIL-R2. The TRAIL-R2-binding agent may be an antibody or functional fragment thereof, including an scFv of a monoclonal antibody specific for TRAIL-R2, or the TRAIL-R2-binding agent may be a ligand of TRAIL-R2, such as TRAIL. The intracellular domain may be further defined as comprising one or more costimulatory domains (e.g., CD28, CD137 (4-1BB), CD134 (OX40), DAP10, DAP12, NKG2D, CD40L, CD27, CD2, CD5, ICAM-1, LFA-1 (CD11a/CD18), Lck, TNFR-I, TNFR-II, Fas, CD30, CD40, 2B4, DNAM, CS1, CD48, NKp30, NKp44, NKp46, NKp80, members of the TNFR superfamily, or a combination thereof) and optionally CD3zeta. The transmembrane domain of the chimeric protein may be from CD28, the alpha chain of the T-cell receptor, beta chain of the T-cell receptor, zeta chain of the T-cell receptor, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, DAP10, or DAP12. In a specific case, the TRAIL-R2-binding agent is an scFv of a monoclonal antibody specific for TRAIL-R2 and the intracellular region comprises the costimulatory domain from 4-1BB. The polynucleotide may or may not be a vector or be in a vector, including any viral vector (e.g., adenoviral vector, lentiviral vector, retroviral vector, or adeno-associated viral vector) or non-viral vector (e.g., plasmid, transposon, nanoparticle, liposome, lipid, metal, or a combination thereof).

In specific embodiments, there are isolated cells comprising any polynucleotide encompassed herein, including eukaryotic or prokaryotic cell and also including human cells. In specific embodiments, the cells are immune cells (T cells including αβ or γδ cells, NK cell, NKT cell, monocyte, macrophage, B cell, or a mixture thereof) or stem cells (mesenchymal stem cell, hematopoietic stem cell, induced pluripotent stem cell, or a mixture or derivative thereof).

In some embodiments, the cell comprises a vector that encodes one or more therapeutic proteins, including at least engineered antigen receptors, such as chimeric antigen receptors (CAR) or transgenic T cell receptors. In some cases, the vector that encodes the CAR and the vector that encodes the chimeric protein are different, whereas in other cases the vector that encodes the CAR and the vector that encodes the chimeric protein are the same vector. The engineered antigen receptor may target a cancer antigen, including one selected from the group consisting of Zona pellucida sperm-binding protein 4 (ZP4), Melanoma-associated antigen D4 (MAGE-D4), CD19, EBNA, CD123, HER2, CA-125, TRAIL/DR4, TRAIL-R2, CD20, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, -2, -8, GAGE-3, -4, -5, -6, -7B, NA88-A, MC1R, mda-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, STATE, hypoxia inducible factors (e.g., HIF-1 and HIF-2), Nuclear Factor-Kappa B (NF-B), Notch receptors (e.g., Notch1-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAGiB, SUNC1, and LRRN1. The CAR may comprise one or more costimulatory domains selected from the group consisting of 4-1BB, CD27, CD28, DAP12, NKG2D, OX-40 (CD134), DAP10, CD40L, 2B4, DNAM, CS1, CD48, NKp30, NKp44, NKp46, NKp80, and a combination thereof. The CAR may comprise a transmembrane domain selected from the group consisting of CD28, the alpha chain of the T-cell receptor, beta chain of the T-cell receptor, zeta chain of the T-cell receptor, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, DAP10, and DAP12.

In one embodiment, there is a method of inhibiting TRAIL-R2-expressing cells in a tumor microenvironment of an individual, comprising the step of administering to the individual a therapeutically effective amount of any cells encompassed herein. The cells may or may not be administered more than once. When administered more than once, the duration of time between administrations may be 1, 2, 3, 4, 5, 6, 7, or more days. In specific cases, the cells are administered in the range of about 10³ to 10¹⁰ cells per dose, including administered intravenously, intradermally, transdermally, intrathecally, intraarterially, intraperitoneally, intranasally, intravaginally, intrarectally, topically, intramuscularly, subcutaneously, mucosally, orally, topically, locally, inhalation, injection, infusion, continuous infusion, localized perfusion bathing target cells directly, via a catheter, via a lavage, in cremes, in lipid compositions, or a combination thereof. In specific embodiments, the individual is administered an additional cancer therapy, such as surgery, radiation, drug therapy, immunotherapy, hormone therapy, or a combination thereof.

In one embodiment, there is a method of treating cancer in an individual, comprising the step of administering to the individual a therapeutically effective amount of any cells encompassed herein. The cells may or may not be administered more than once. When administered more than once, the duration of time between administrations may be 1, 2, 3, 4, 5, 6, 7, or more days. In specific cases, the cells are administered in the range of about 10³ to 10¹⁰ cells per dose, including administered intravenously, intradermally, transdermally, intrathecally, intraarterially, intraperitoneally, intranasally, intravaginally, intrarectally, topically, intramuscularly, subcutaneously, mucosally, orally, topically, locally, inhalation, injection, infusion, continuous infusion, localized perfusion bathing target cells directly, via a catheter, via a lavage, in cremes, in lipid compositions, or a combination thereof. In specific embodiments, the individual is administered an additional cancer therapy, such as surgery, radiation, drug therapy, immunotherapy, hormone therapy, or a combination thereof.

In specific embodiments, there are methods of enhancing a cellular therapy for cancer in an individual, comprising the step of modifying cells of the cellular therapy to express any polynucleotide encompassed herein. Such cells of the cellular therapy may express one or more chimeric antigen receptors.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIGS. 1A-1D. The novel TR2.4-1BB co-stimulatory receptor induces downstream signaling. (1A) MUC1 expression on TNBC cell lines BT-20, MDA-MB-231 and MUC1− cell line 293T (1B) in vitro cytolytic function of control (NT) and CAR.MUC1 T cells assessed in a 5 hr ⁵¹Cr release assay at effector:targets of 5:1 to 40:1 using MUC1+ targets (BT-20, MDA-MB-231) and MUC1-target (293T). Data represents mean±SEM (n=5). (1C) Schematic representation of TR2.41BB construct (1D) NT and TR2.4-1BB transduced T cells were cultured alone or in the presence of rTR2 or anti-CD3 and anti-CD28. Cells were harvested at 0, 60 and 120 minutes, the nuclear fraction was harvested, and an ELISA was performed to measure the translocation of NFkB into the nucleus. Data represents mean±SEM (n=3).

FIGS. 2A-2D. In vivo generated myeloid derived suppressor cells (MDSCs) of the monocytic lineage demonstrated suppressive potential. (2A) MDSCs were harvested after 7 days of culture with GM-CSF and IL-6. Flow cytometry was performed to validate the presence of CD33+CD11b+CD14+ HLA-DRlow TR2+ cells (2B) MDSCs were co-cultured with autologous CFSE-labeled T cells at a 1:1 and 1:2 ratio in the presence of T cell stimuli. After 3 days T cell proliferation was measured as CFSE-dilution using flow cytometry (n=15) (2C) quantification of T cell proliferation (2D) Supernatants were collected from suppression assay culture and ELISA was performed for IFNγ. Data represents mean±SEM (n=2).

FIGS. 3A-3E. Introduction of MDSCs into a TNBC cell derived xenograft (CDX) mouse model leads to a higher tumor burden and a marked increase in tumor volume. (3A) Schematic of in vivo experiment where NSG mice were transplanted with GFP-FFLuc labelled SUM-159 cells with or without MDSCs (3B) Bioluminescence data and (3C) quantification from mice assessed weekly to monitor tumor burden and (3D) tumor volume measured by calipers (3E) tumor weight quantification (mean±SEM, n=5/group)

FIGS. 4A-4D. MDSCs stimulate tumor growth in vivo by promoting vascularization and accumulation of fibroblasts. (4A) Tumor sections from mice transplanted with SUM-159 cells in presence or absence of MDSCs stained with CD31 Ab. Total of 10 fields of view were analyzed for each group, representative images and (4B) quantification of CD31+ staining. (4C) Tumor sections from mice transplanted with SUM-159 cells in presence or absence of MDSCs were stained with αSMA Ab for fibroblasts. Total of 14 fields of view were analyzed for each group, representative images and (4D) quantification of aSMA+ staining. Data represents mean±SEM (n=7).

FIGS. 5A-5B. MDSCs persist at the tumor site for 14 days. (5A) NSG mice co-injected with SUM-159 cells and MDSCs were sacrificed on 7, 14 and 21 days, flow cytometry was performed to assess the presence of MDSCs at tumor site, (5B) quantification of MDSC numbers.

FIGS. 6A-6D. TRAIL-R2.4-1BB construct is able to rescue the cytotoxic activity of CAR.MUC1 in the presence of MDSCs. (6A) Transgenic expression of both CAR.MUC1 and TRAIL-R2.4-1BB T cells detected using anti-IgG2-CH3 spacer and biotinylated TR2 respectively (6B) CAR MUC1 T cells cultured with BT-20 (MUC1+) cells in presence or absence of MDSCs and cytotoxic function was assessed in a 5 hr 51Cr-release assay. Data represents mean±SEM (n=7) (6C) CAR.MUC1, TR2.41BB and CAR. MUC1. TRAIL-R2.4-1BB T cells cultured with BT-20 and 51Cr labelled MDSCs and percentage of MDSC lysis was assessed in a 5 hr 51Cr-release assay. Data represents mean±SEM (n=5) (6D) CAR.MUC1, TR2.41BB and CAR. MUC1. TR2.41BB T cells cultured with 51Cr labelled BT-20 targets in presence of MDSCs and cytotoxic function was assessed in a 5 hr 51Cr-release assay. Data represents mean±SEM (n=5).

FIGS. 7A-7D. Expression of TRAIL-R2.4-1BB enhances expansion and persistence of CAR.MUC1 T cells and augments their anti-tumor activity in vivo whilst the presence of MDSCs. (7A) Schematic of in vivo experiment where NSG mice transplanted with MDA-MB-231 with or without MDSCs treated with GFP-FFLuc labelled NT, CAR.MUC1, TR2.41BB or CAR.MUC1.TR2.41BB T cells (7B) Bioluminescence imaging indicating expansion and persistence of T cells and (7C) quantification of bioluminescence at tumor site (7D) tumor volume was measured by calipers. Mean±SEM, n=4/group.

FIGS. 8A-8B. Superior killing of SUM-159 tumor cells and decreased metastasis despite presence of MDSCs by CAR.MUC1.TR2.41BB (8A) Schematic of in vivo experiment where NSG mice transplanted with GFP-FFLuc labelled SUM-159 with or without MDSCs treated with NT, CAR.MUC1, TR2.41BB or CAR.MUC1.TR2.41BB T cells (8B) volume was measured by calipers. Mean±SEM, n=5/group.

FIGS. 9A-9D. Combined expression of TR2.41BB and CAR.HER2 on T cells enhances anti-tumor potential in a HER2+BC model. (9A) CAR.HER2 T cells cultured with MDA-MB-453 (HER2+) cells in presence or absence of MDSCs and cytotoxic function was assessed in a 5 hr 51Cr-release assay. Data represents mean±SEM (n=3) (9B) CAR.HER2, TR2.41BB and CAR.HER2. TR2.41BB T cells cultured with 51Cr labelled MDA-MB-453 in presence of MDSCs and cytotoxic function was assessed in a 5 hr 51Cr-release assay. Data represents mean±SEM (n=3). (9C) NSG mice were transplanted with GFP-FFLuc labelled MDA MB-453 cells with or without MDSCs, tumor volume measured by calipers (mean±SEM, n=3-5/group) (9D) NSG mice transplanted with MDA-MB-453 with or without MDSCs treated with GFP-FFLuc labelled NT, CAR.HER2, TR2.41BB or CAR.HER2.TR2.41BB T cells and tumor volume was measured by calipers (mean±SEM, n=5/group).

FIGS. 10A-10B. Resting and activated T cells show high surface expression of TR2. Resting and activated PBMCs were harvested at 24, 48 and 72 hrs and flow cytometry was performed to assess TR2 expression on CD4+, CD8+ T cells and NK cells (10A) representative flow gating, (10B) TR2 quantification, mean±SEM, (n=3).

FIGS. 11A-11E. TR2.41BB does not induce toxicity in resting and activated CD4+ and CD8+ T cells owing to increased expression of cFLIP. Resting and activated T cells (72 hr) were labelled with CFSE and co-cultured in the presence of T cells transduced with either CAR.MUC1 or TR2.41BB constructs or both. Cells were harvested after 6 hr and 24 hr, flow cytometry was performed using 7-AAD and annexin to assess the percentage of apoptosis (11A) representative flow gating, percentage of apoptosis in (11B) resting and (11C) activated CD4+ and CD8+ T cells, mean±SEM, (n=3-4). (11D) Protein lysate from resting T cells, activated T cells, and MDSCs was collected, and western blot was performed (11E) quantification. The membrane was imaged on Licor, and cFLIP expression was normalized to the internal control, GAPDH (n=1).

FIG. 12. Generation of human MDSCs in vitro. PBMCs were obtained from healthy volunteers, CD14+ cells were isolated and were then cultured for 7 days in presence of GM-CSF and IL-6. GM-CSF was added on day 1, 3 and 5, whereas IL-6 was added only on day 5 and then harvested a week later.

FIGS. 13A-13C. The novel TRAIL-R2.4-1BB co-stimulatory receptor specifically targets TRAIL-R2 (13A) CRISPR knockout of TRAIL-R2 in K562 cell line (13B) Schematic representation of truncated TRAIL-R2 construct (13C) in vitro cytolytic function of control (NT) and CAR.TRAIL-R2.4-1BB T cells assessed in a 6 hr 51Cr release assay at effector:targets of 5:1 to 40:1 using truncated TRAIL-R2 (lacking death domain) expressing K562 cells as targets.

FIG. 14. Metastasis to lungs tends to increase in presence of MDSCs. NSG mice transplanted with GFP-FFLuc labelled SUM-159 cells with or without MDSCs. Mice were sacrificed at the end of the study (day 42), organs were harvested and imaged for metastatic spread, representative images shown, n=4-5.

FIGS. 15A-15D. MUC1.TRAIL-R2.4-1BB CARTs show similar memory phenotype and exhaustion/activation status compared to CAR.MUC1 or TRAIL-R2.4-1BB cells alone. Activated T cells were either transduced with CAR.MUC1 or TRAIL-R2.4-1BB constructs alone or sequentially with CAR.MUC1 and TRAIL-R2.4-1BB, flow cytometry was performed on day 14 after transduction to assess (15A) memory phenotype, (15B) exhaustion markers, (15C) activation markers, and (15D) CAR expression. Data represents mean±SEM, (n=3).

FIG. 16. MUC1.TRAIL-R2.4-1BB CARTs prevent tumor metastasis. NSG mice transplanted with GFP-FFLuc labelled SUM-159 cells with or without MDSCs treated with NT, CAR.MUC1, TRAIL-R2.4-1BB or CAR.MUC1.TRAIL-R2.4-1BB T cells. Mice were sacrificed at the end of the study (day 49), organs were harvested and imaged for metastatic spread, representative images shown, n=4-5.

### DETAILED DESCRIPTION

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In specific embodiments, a vector is engineered through recombinant nucleic acid technologies, and a cell is engineered through transfection or transduction of an engineered vector.

The term “heterologous” as used herein refers to genes or their gene products that are not normally part of a genetic material, or products thereof, in a host organism. A heterologous gene encoding a heterologous protein may be integrated into the host DNA, causing permanent expression, or may not be integrated, resulting in transient expression.

The term “subject,” as used herein, generally refers to an individual having a biological sample that is undergoing processing or analysis and, in specific cases, has or is suspected of having cancer. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as benign or malignant neoplasias, or cancer. The subject may being undergoing or having undergone treatment. The subject may be asymptomatic. The subject may be healthy individuals but that are desirous of prevention of cancer. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

Embodiments of the disclosure concern methods and compositions for selectively targeting cells in the TME that express TRAIL-R2. The targeting of such cells results in a reduction at least in part of immune suppression in the TME via apoptosis of the TRAIL-R2-expressing cells, thereby directly or indirectly leading to enhanced ability to kill cancer cells located in the TME.

### I. TRAIL-R2-Targeting Chimeric Proteins and Compositions Related Thereto

The disclosure concerns reagents that relate to chimeric proteins that are able to target or bind TRAIL-R2, including in certain cells in the TME, such as to directly or indirectly cause apoptosis of cells expressing the TRAIL-R2. In specific embodiments, the chimeric protein is a cell surface protein and comprises at least (1) an extracellular region comprising a TRAIL-R2-binding agent; and (2) an intracellular region that is not from TRAIL-R2. The chimeric protein in specific embodiments includes a transmembrane domain that may or may not be from TRAIL-R2 itself. The extracellular region may be of any kind or source, including an antibody or functional fragment thereof or another kind of protein, such as a natural or non-natural ligand of TRAIL-R2. In specific embodiments, the TRAIL-R2-binding agent of the extracellular region comprises one or more antibodies or functional fragments thereof, including that are from a monoclonal antibody, such as an scFv of a monoclonal antibody specific for TRAIL-R2, although other antibody types may be utilized. In alternative embodiments, the TRAIL-R2-binding agent comprises one or more ligands of TRAIL-R2. The ligand may be known in nature to be a ligand of TRAIL-R2, or the ligand may be generated by the hand of man. In specific examples, the ligand of TRAIL-R2 is TRAIL. In specific cases the entire TRAIL-R2 ligand is not utilized but only a region that physically binds to TRAIL-R2.

The intracellular region of the TRAIL-R2-targeting chimeric protein may be of any kind, but in specific embodiments the intracellular region comprises at least one domain that is capable of an activity that facilitates function of cells that express the chimeric protein. In some cases, the intracellular region is selected for the purpose of enhancing expansion, persistence, and/or cytotoxicity of cells that express the chimeric protein. Specific aspects of the chimeric protein include one or more costimulatory regions that enhance at least expansion and persistence of the cells expressing the chimeric protein. In some cases only one costimulatory domain is used, whereas in other cases more than one co-stimulatory domain is used. Examples of costimulatory domains include those from 4-1BB, CD27, CD28, DAP12, NKG2D, OX-40 (CD134), DAP10, CD40L, 2B4, DNAM, CS1, CD48, NKp30, NKp44, NKp46, NKp80, or a combination thereof. In a particular case, the costimulatory domain is from 4-1BB.

The TRAIL-R2-targeting chimeric protein is a membrane bound protein and as such comprises a transmembrane domain. The transmembrane domain may be from any protein, but in specific cases it is from CD28, the alpha chain of the T-cell receptor, beta chain of the T-cell receptor, zeta chain of the T-cell receptor, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, DAP10, or DAP12.

In particular embodiments, the TRAIL-R2-targeting chimeric protein in the cells comprises an extracellular spacer domain that links the TRAIL-R2 extracellular region and the transmembrane domain. Extracellular spacer domains, which may also be referred to as hinges, may include, but are not limited to, Fc fragments of antibodies or fragments or derivatives thereof, hinge regions of antibodies or fragments or derivatives thereof, CH2 regions of antibodies, CH3 regions of antibodies, artificial spacer sequences or combinations thereof. Examples of extracellular spacer domains include but are not limited to IgG2-CH3 hinge, CD8-alpha hinge, CD28, artificial spacers made of polypeptides such as Gly3, or CH1, CH3 domains of IgGs (such as human IgG1 or IgG4). In specific cases, the extracellular spacer domain may comprise (i) a hinge, CH2 and CH3 regions of IgG4, (ii) a hinge region of IgG4, (iii) a hinge and CH2 of IgG4, (iv) a hinge region of CD8-alpha, (v) a hinge, CH2 and CH3 regions of IgG1, (vi) a hinge region of IgG1 or (vii) a hinge and CH2 of IgG1, (viii) a hinge region of CD28, or a combination thereof. In specific embodiments, the hinge is from IgG1 and in certain aspects the CAR polypeptide comprises a particular IgG1 hinge amino acid sequence.

In specific embodiments, a polynucleotide comprises an expression construct that encodes the TRAIL-R2-targeting chimeric protein. In particular cases, the polynucleotide is a vector, including a viral vector or a non-viral vector. In cases wherein the vector is a viral vector, the viral vector may be an adenoviral vector, lentiviral vector, retroviral vector, or adeno-associated viral vector. In cases wherein the vector is a non-viral vector, the the non-viral vector may comprise a plasmid, transposon, nanoparticle, liposome, lipid, metal, or a combination thereof. Any polynucleotide encompassed herein may be produced by standard recombinant technology. The polynucleotide may be provided in a kit, and/or one or more primers to generate at least part of the polynucleotide may be provided in a kit.

In particular embodiments, the polynucleotide is comprised in a cell, including an isolated cell, and under suitable conditions the TRAIL-R2-targeting chimeric protein may be expressed from the expression construct on the polynucleotide in the cell. The cell housing the vector may be of any kind, including a eukaryotic or prokaryotic cell. In particular embodiments, the cell is a mammalian cell and including a human, mouse, or rat cell. In particular embodiments, the cells are suitable for use as cellular therapy, including for adoptive cell therapy. The cells may be autologous or allogeneic with respect to a recipient individual. In specific embodiments, the cell is an immune cell of any kind, a stem cell of any kind, derivatives thereof, or a mixture thereof. In cases wherein the immune cell expresses the chimeric protein, the immune cell may be a T cell, NK cell, NKT cell, monocyte, macrophage, B cell, derivatives thereof, or a mixture thereof. In cases wherein the cell expressing the chimeric protein is a T cell, the T cell may be of any kind, including at least an αβ T cell or a γδ T cell. In cases wherein the cell is a stem cell, the stem cell may be of any kind, including at least a mesenchymal stem cell, hematopoietic stem cell, induced pluripotent stem cell, or a mixture or derivative thereof.

In certain embodiments, the cells express a heterologous protein that is not the chimeric protein, and in specific embodiments one or more heterologous proteins other than the chimeric protein are expressed from the cell. In some cases, the heterologous protein(s) are present in the cell on a vector, including expressed from an expression construct. The vector that encodes the chimeric protein and the vector that encodes the heterologous protein(s) may or may not be the same. In specific embodiments, when the chimeric protein and another protein are co-expressed from the same molecule, they may be separated by a 2A sequence of any kind or by IRES, for example. In a 5′ to 3′ orientation along the expression construct the chimeric protein may come first or subsequent to any heterologous protein.

In specific embodiments, the cells may be housed in a suitable repository, and the cells may be autologous with respect to a recipient or allogeneic with respect to a recipient. The repository may be one in which cells are preserved for future use in any manner, but in specific cases the cells are cryopreserved. In such cases, the cells may be frozen in an medium comprising one or more cryoprotectants, such as glycerol, DMSO, propylene glycol, ethylene glycol, mixtures thereof, and so forth. In some embodiments, the cells are modified to express the chimeric protein, are housed in a suitable repository following the modification, and then are obtained from the repository upon a need. In some cases, the cells are housed in the repository after or before further modifications, such as upon modification to express one or more heterologous proteins. In specific cases, the cells are housed in the repository after modification to express the chimeric protein, and following thawing the cells are further modified to tailor the cells to express one or more therapeutic proteins specifically suited for an individual in need, such as modified to express one or more CARs that target a cancer antigen of a cancer of the individual.

### II. Methods Related to TRAIL-R2-Targeting Chimeric Proteins

Embodiments of the disclosure include methods of using TRAIL-R2-targeting chimeric proteins. The proteins comprise an extracellular region that is configured to be able to bind TRAIL-R2, and so any method for which the binding of the chimeric protein to TRAIL-R2 is useful is encompassed herein. In specific embodiments, there are methods of using TRAIL-R2-targeting chimeric proteins to inhibit cells expressing TRAIL-R2 for any purpose, including any therapeutic purpose. The cells that are being inhibited by the TRAIL-R2-targeting chimeric protein may be able to impede activity of therapeutic cells, and so upon binding of the TRAIL-R2-targeting chimeric proteins to TRAIL-R2, the cells that express TRAIL-R2 may be inhibited in any activity. In specific embodiments, the cells expressing TRAIL-R2 undergo apoptosis following binding of the TRAIL-R2-targeting chimeric protein to TRAIL-R2. In specific cases, the cells are MDSCs.

In particular embodiments, there are methods of inhibiting cells in a tumor microenvironment (TME), including cells in the TME that express TRAIL-R2. Cells in the TME, including at least MDSCs, are bound by cells expressing the TRAIL-R2-targeting chimeric protein and, as a result, use of the cells expressing the TRAIL-R2-targeting chimeric protein in the TME results in inhibition, including death, of cells in the TME that express TRAIL-R2. In specific embodiments, inhibiting cells in the TME allows a reduction of immune suppression in the TME and provides an environment where therapeutic cells that are present in the TME have an increased efficacy to kill cancer cells therein or near to the TME. In effect, use of cells that express the TRAIL-R2-targeting chimeric protein have an enhanced efficacy to inhibit cells in the TME compared to cells that lack the TRAIL-R2-targeting chimeric protein. Methods include administering to a TME, either systemically or locally, an effective amount of cells that express the chimeric protein.

Included herein are methods of inhibiting MDSCs in a tumor microenvironment in an individual, comprising the step of administering to the individual a therapeutically effective amount of cells expressing at least the TRAIL-R2-targeting chimeric protein. The cells expressing the TRAIL-R2-binding chimeric protein are able to bind the MDSCs that naturally express the TRAIL-R2 receptor, that ultimately thereby initiates apoptosis for the MDSCs. MDSCs that are known to promote tumor growth, metastasis and immune suppression are thus inhibited by compositions comprising cells that express the chimeric protein, including inhibition of the MDSCs by the chimeric protein-expressing cells at the intractable TME. In some embodiments, the presence of MDSCs are determined as part of the methods, such as by assaying for one or more MDSC-specific markers. In specific cases, flow cytometry is utilized to determine expression of multiple markers on MDSCs.

Embodiments of the disclosure include methods of treating cancer in an individual that is known to have cancer or suspected of having cancer. The individual may be in need of preventing cancer, in need of delaying the onset of cancer, in need of reducing the severity of one or more symptoms of cancer, and/or in need of preventing or delaying metastasis of cancer, and so forth. The methods in specific cases comprise the step of administering to the individual a therapeutically effective amount of cells that express the TRAIL-R2-targeting chimeric protein.

In one embodiment, there is a method of enhancing a cellular therapy for cancer in an individual, comprising the step of modifying cells of a cellular therapy to express a TRAIL-R2-binding chimeric protein. In specific embodiments, prior to administration of a therapeutically effective amount to an individual with cancer, therapeutic cells of any kind are modified to express the TRAIL-R2-targeting chimeric protein. The cells may or may not have been otherwise modified, such as to express one or more heterologous proteins.

In particular embodiments, there are methods of overcoming a poor response to a therapy of any kind, including a cellular therapy, such as cellular therapy that can be inhibited or that is inhibited at least in part by the TME and/or MDSCs, including MDSCs that express TRAIL-R2. In alternative cases, the poor response includes a cancer therapy other than a cellular therapy. Any methods herein may reduce the severity of one or more symptoms of cancer upon administering a therapeutically effective amount of cells expressing the chimeric protein to an individual that has cancer. Methods herein encompass administering a therapeutically effective amount of cells that express the TRAIL-R2-targeting chimeric protein to reduce tumor load in an individual. Methods of the disclosure also encompass methods of preventing or delaying onset of metastasis in an individual, comprising the step or providing to an individual with cancer that has not yet metastasized, or that has not been known to metastasize, a therapeutically effective amount of cells expressing the TRAIL-R2-targeting chimeric protein.

For any method encompassed herein, the cells of the cell therapy that express the chimeric protein optionally also express one or more heterologous proteins, including therapeutic proteins. The heterologous proteins may be used in the method because they increase efficacy of the cells themselves as a therapeutic moiety (such as when a CAR molecule can increase expansion and persistence for the cells), and/or the heterologous proteins themselves are therapeutic.

In any methods of use herein, the therapeutic cells expressing at least the chimeric proteins are provided to an individual in need thereof by any suitable means, including at least locally or systemically. In specific cases, the cells are delivered directly to the TME in an individual, or they may be delivered systemically to the individual.

### III. Heterologous Proteins Including Chimeric Antigen Receptors

In particular embodiments, the cells of the disclosure that express the TRAIL-R2-targeting chimeric protein also express one or more heterologous proteins. The heterologous protein may act independently of the chimeric protein and is a separate molecule from the chimeric protein, in specific cases. The heterologous protein may be co-expressed with the chimeric protein from the same vector, whether or not their expression is regulated by the same regulatory element(s). In other cases the chimeric protein and heterologous protein(s) may be expressed from different vectors, and in such cases the cells may or may not have been transfected or transduced with the different vectors at the same time.

In specific cases, the heterologous protein enhances the therapeutic efficacy or potential of the cells. The heterologous protein may enhance the ability of the TRAIL-R2-targeting chimeric protein to be effective. The heterologous protein may enhance the ability of the cells to be effective. The heterologous protein may enhance the ability of the TRAIL-R2-targeting chimeric protein and the cells to be effective. In specific cases the therapeutic protein is chimeric itself.

In specific embodiments, the heterologous protein is an engineered antigen receptor. The engineered antigen receptor may be of any kind, but in specific embodiments it is a chimeric antigen receptor, including one that targets any antigen, such as any cancer antigen. Other heterologous proteins include cytokines, cytokine receptors, transgenic T cell receptors, a dominant negative TGFβ receptor type II (TGFβ-DNR), a CAR that targets tumor vasculature (such as VEGFR-2-specific CARs), chimeric IL-4 receptors (signal converters such as comprising the ectodomain of the IL-4 receptor and the endodomain of the IL-7Rα), or a combination thereof.

A. Chimeric Antigen Receptors

The present disclosure provides for cells (including immune cells) that express a TRAIL-R2-binding chimeric protein and also a chimeric antigen receptor (CAR). In some cases, one or more CARs are expressed by the cells, and the CAR may be first generation, second generation, or third or a subsequent generation, for example. The CAR may or may not be bispecific for two or more different antigens, or trispecific for three or more different antigens, for example. The CAR may comprise an extracellular domain comprising one or more antigen binding domains; a transmembrane domain; and an intracellular domain comprising one or more co-stimulatory domains. Each costimulatory domain may comprise the co-stimulatory domain of any one or more of, for example, members of the TNFR superfamily, CD28, CD137 (4-1BB), CD134 (OX40), DAP10, DAP12, NKG2D, CD40L, CD27, CD2, CD5, ICAM-1, LFA-1 (CD11a/CD18), Lck, TNFR-I, TNFR-II, Fas, CD30, CD40, 2B4, DNAM, CS1, CD48, NKp30, NKp44, NKp46, NKp80, or combinations thereof, for example. In specific embodiments, the CAR comprises CD3zeta. In certain embodiments, the CAR lacks one or more specific co-stimulatory domains; for example, the CAR may lack 4-1BB and/or lack CD28.

In particular embodiments, the CAR polypeptide in the cells comprises an extracellular spacer domain that links the antigen binding domain and the transmembrane domain. Extracellular spacer domains may also be referred to as hinges and may include, but are not limited to, Fc fragments of antibodies or fragments or derivatives thereof, hinge regions of antibodies or fragments or derivatives thereof, CH2 regions of antibodies, CH3 regions antibodies, artificial spacer sequences or combinations thereof. Examples of extracellular spacer domains include but are not limited to CD8-alpha hinge, CD28, artificial spacers made of polypeptides such as Gly3, or CH1, CH3 domains of IgGs (such as human IgG1 or IgG4). In specific cases, the extracellular spacer domain may comprise (i) a hinge, CH2 and CH3 regions of IgG4, (ii) a hinge region of IgG4, (iii) a hinge and CH2 of IgG4, (iv) a hinge region of CD8-alpha, (v) a hinge, CH2 and CH3 regions of IgG1, (vi) a hinge region of IgG1 or (vii) a hinge and CH2 of IgG1, (viii) a hinge region of CD28, or a combination thereof. In specific embodiments, the hinge is from IgG1.

The transmembrane domain in some embodiments is derived either from a natural or from a synthetic source. Where the source is natural, the domain in some aspects is derived from any membrane-bound or transmembrane protein. The transmembrane domain may or may not be derived from the same protein as a costimulatory domain of the CAR. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, and DAP molecules, such as DAP10 or DAP12. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine may be found at each end of a synthetic transmembrane domain.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA. It is contemplated that the chimeric construct can be introduced into immune cells of any kind as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression. Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV. Non-viral vectors include plasmids, transposons, nanoparticles, liposome, lipids, metals, or a combination thereof.

In specific cases, a CAR targets one or more particular antigens, including cancer antigens that may be associated with a particular type of cancer. The antigen may be associated with a solid tumor or with a hematological cancer. In specific cases, the CAR targets a cancer antigen selected from the group consisting of Zona pellucida sperm-binding protein 4 (ZP4), Melanoma-associated antigen D4 (MAGE-D4), CD19, CD70, EBNA, CD123, HER2, CA-125, TRAIL-R1/DR4, TRAIL-R2DR5, CD20, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD7, CD23, CD30, HERV-K, IL-11Ralpha, Tem8, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, -2, -8, GAGE-3, -4, -5,-6, -7B, NA88-A, MC1R, mda-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, STATE, hypoxia inducible factors (e.g., HIF-1 and HIF-2), Nuclear Factor-Kappa B (NF-B), Notch receptors (e.g., Notch1-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAGiB, SUNC1, and LRRN1.

In particular embodiments, the CAR targets TRAIL-R2. In such cases, there is a TRAIL-R2-targeting chimeric protein and also a TRAIL-R2 CAR, including that may be co-expressed from the same vector. In such an example, the TRAIL-R2 targeting chimeric protein lacks CD3zeta, but the TRAIL-R2 CAR includes CD3zeta or a suitable substitute.

B. T Cell Receptors

In some embodiments, genetically engineered antigen receptors include recombinant TCRs and/or TCRs cloned from naturally occurring T cells. A “T cell receptor” or “TCR” refers to a molecule that contains variable a and 3 chains (also known as TCRα and TCRβ, respectively) or variable 7 and S chains (also known as TCRγ and TCRδ, respectively) and that is capable of specifically binding to an antigen peptide bound to a MHC receptor. In some embodiments, the TCR is in the αβ form. The TCR may target any antigen referred to elsewhere herein.

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable 3 chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., a-chain constant domain or Ca, typically amino acids 117 to 259 based on Kabat, 3-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

C. Cytokines

In some embodiments, the cells are engineered to express one or more heterologous cytokines and/or are engineered to upregulate normal expression of one or more heterologous cytokines. The cells may or may not be transduced or transfected for one or more cytokines on the same vector as other genes.

One or more cytokines may be co-expressed from the vector as a separate polypeptide from the antigen receptor. Interleukin-15 (IL-15), for example, is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits activation-induced cell death (AICD). In addition to IL-15, other cytokines are envisioned. These include, but are not limited to, cytokines, chemokines, and other molecules that contribute to the activation and proliferation of cells used for human application. NK cells expressing IL-15 are capable of continued supportive cytokine signaling, which is useful for their survival post-infusion.

In specific embodiments, the cells expresses one or more exogenously provided cytokines. As one example, the cytokine is IL-7, IL-15, IL-12, IL-2, IL-18, IL-21, GMCSF, or a combination thereof. The cytokine may be exogenously provided to the NK cells because it is expressed from an expression vector within the cell. In an alternative case, an endogenous cytokine in the cell is upregulated upon manipulation of regulation of expression of the endogenous cytokine, such as genetic recombination at the promoter site(s) of the cytokine. In cases wherein the cytokine is provided on an expression construct to the cell, the cytokine may be encoded from the same vector as a suicide gene and/or as the TRAIL-R2-targeting chimeric protein.

D. Suicide Genes

In particular embodiments, a suicide gene is utilized in conjunction with the TRAIL-R2-targeting cell therapy to control its use and allow for termination of the cell therapy at a desired event and/or time. The suicide gene may be employed in transduced cells for the purpose of eliciting death for the transduced cells when needed. The cells of the present disclosure that have been modified to harbor a vector encompassed by the disclosure may comprise one or more suicide genes. In some embodiments, the term “suicide gene” as used herein is defined as a gene which, upon administration of a prodrug or other agent, effects transition of a gene product to a compound which kills its host cell. In other embodiments, a suicide gene encodes a gene product that is, when desired, targeted by an agent (such as an antibody) that targets the suicide gene product, and in some cases such a system is referred to as a safety switch.

In some cases, the cell therapy may be subject to utilization of one or more suicide genes of any kind when an individual receiving the cell therapy and/or having received the cell therapy shows one or more symptoms of one or more adverse events, such as cytokine release syndrome, neurotoxicity (including at least immune effector cell-associated neurotoxicity syndrome), anaphylaxis/allergy, and/or on-target/off tumor toxicities (as examples) or is considered at risk for having the one or more symptoms, including imminently. The use of the suicide gene may be part of a planned protocol for a therapy or may be used only upon a recognized need for its use. In some cases the cell therapy is terminated by use of agent(s) that targets the suicide gene or a gene product therefrom because the therapy is no longer required.

Utilization of the suicide gene may be instigated upon onset of at least one adverse event for the individual, and that adverse event may be recognized by any means, including upon routine monitoring that may or may not be continuous from the beginning of the cell therapy. The adverse event(s) may be detected upon examination and/or testing. In cases wherein the individual has cytokine release syndrome (which may also be referred to as cytokine storm), the individual may have elevated inflammatory cytokine(s) (merely as examples: interferon-gamma, granulocyte macrophage colony-stimulating factor, IL-10, IL-6 and TNF-alpha); fever; fatigue; hypotension; hypoxia, tachycardia; nausea; capillary leak; cardiac/renal/hepatic dysfunction; or a combination thereof, for example. In cases wherein the individual has neurotoxicity, the individual may have confusion, delirium, aplasia, and/or seizures. In some cases, the individual is tested for a marker associated with onset and/or severity of cytokine release syndrome, such as C-reactive protein, IL-6, TNF-alpha, and/or ferritin

Examples of suicide genes include engineered nonsecretable (including membrane bound) tumor necrosis factor (TNF)-alpha mutant polypeptides (see PCT/US19/62009, which is incorporated by reference herein in its entirety), and they may be affected by delivery of an antibody that binds the TNF-alpha mutant. Examples of suicide gene/prodrug combinations that may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be utilized. Other suicide genes include CD20, CD52, inducible caspase 9, purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP), as examples.

In particular embodiments, vectors that encode the TRAIL-R2-targeting chimeric protein, or any vector encompassed herein, include one or more suicide genes. The suicide gene may or may not be on the same vector as the chimeric protein and/or a heterologous protein(s). In cases wherein the suicide gene is present on the same vector as the chimeric protein and/or the heterologous protein(s), the suicide gene and the chimeric protein and/or heterologous protein(s) may be separated by an IRES or 2A element, for example.

### IV. Pharmaceutical Compositions

Pharmaceutical compositions of the present disclosure comprise an effective amount of cells expressing the TRAIL-R2-targeting chimeric protein dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that comprises cells expressing the TRAIL-R2-targeting chimeric protein will be known to those of skill in the art in light of the present disclosure, as exemplified by Remington: The Science and Practice of Pharmacy, 21st Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The pharmaceutical compositions may comprise different types of carriers depending on whether it is to be administered in solid, liquid or aerosol form, and whether it need to be sterile for such routes of administration as injection. The presently disclosed compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The cells expressing the TRAIL-R2-targeting chimeric protein may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the compositions of the present disclosure suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may concern the use of a pharmaceutical lipid vehicle compositions that include cells expressing the TRAIL-R2-targeting chimeric protein and optionally an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds that contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the cells expressing the TRAIL-R2-targeting chimeric protein may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In particular embodiments of the present disclosure, the cells expressing the TRAIL-R2-targeting chimeric protein are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations that are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, compositions may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,613,308; 5,466,468; 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other particular embodiments of the disclosure, the active compound cells expressing the TRAIL-R2-targeting chimeric protein may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### V. Combination Therapies

In certain embodiments, the compositions and methods of the present embodiments involve a cancer therapy that is additional to the compositions comprising cells expressing the TRAIL-R2-targeting chimeric protein. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, hormone therapy, or a combination of the foregoing. The additional therapy may be in the form of adjuvant or neoadjuvant therapy.

In some embodiments, the additional therapy is the administration of small molecule enzymatic inhibitor(s) or anti-metastatic agent(s). In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent(s). The additional therapy may be one or more of the chemotherapeutic agents known in the art.

An immune cell therapy (in addition to the cell therapy of the disclosure) may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from the composition(s) of the disclosure, one would generally ensure that a significant period of time did not expire between the time of each delivery, such that the two compounds would still be able to exert an advantageously combined effect on the patient. In such instances, it is contemplated that one may provide a patient with the immunotherapy therapy and the disclosed compositions within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Administration of any compound or cell therapy of the present embodiments to a patient will follow general protocols for the administration of such compounds, taking into account the toxicity, if any, of the agents. Therefore, in some embodiments there is a step of monitoring toxicity that is attributable to combination therapy.

A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above.

B. Radiotherapy

Other factors that cause DNA damage and have been used extensively include what are commonly known as 7-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

C. Immunotherapy

The skilled artisan will understand that additional immunotherapies (outside of the disclosed cell therapy) may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells other than those having knockdown or knockout of TGF-beta R2.

Antibody-drug conjugates have emerged as a breakthrough approach to the development of cancer therapeutics. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons of any kind, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

E. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VI. Vectors

The TRAIL-R2-targeting chimeric protein may be delivered to the recipient cell by any suitable vector, including by a viral vector or by a non-viral vector. Examples of viral vectors include at least retroviral, lentiviral, adenoviral, or adeno-associated viral vectors. Examples of non-viral vectors include at least plasmids, transposons, lipids, nanoparticles, liposomes, combinations thereof, and so forth.

In cases wherein the cell is transduced with a vector encoding the TRAIL-R2-targeting chimeric protein and also requires transduction or transfection of another gene or genes into the cell, such as a CAR and/or another heterologous protein, they may or may not be comprised on or with the same vector. In some cases, the TRAIL-R2-targeting chimeric protein and the heterologous protein are expressed from the same vector molecule, such as the same viral vector molecule. In such cases, their expression may or may not be regulated by the same regulatory element(s). When the TRAIL-R2-targeting chimeric protein and the heterologous proteins are on the same vector, they may or may not be expressed as separate polypeptides. In cases wherein they are expressed as separate polypeptides, they may be separated on the vector by a 2A element or IRES element (or both kinds may be used on the same vector once or more than once), for example.

One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure.

A. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells may be comprised of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters, for example. In cases wherein the vector is utilized for the generation of cancer therapy, a promoter may be effective under conditions of hypoxia.

B. Promoter/Enhancers

The expression constructs provided herein comprise a promoter to drive expression of the antigen receptor and other cistron gene products. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, for example, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at GenBank®, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

C. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

As detailed elsewhere herein, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the equine rhinitis A virus (E2A) or the F2A (Foot-and-mouth disease virus 2A) or a “2A-like” sequence (e.g., Thosea asigna virus 2A; T2A) or porcine teschovirus-1 (P2A). In specific embodiments, in a single vector the multiple 2A sequences are non-identical, although in alternative embodiments the same vector utilizes two or more of the same 2A sequences. Examples of 2A sequences are provided in US 2011/0065779 which is incorporated by reference herein in its entirety.

D. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

E. Selection and Screenable Markers

In some embodiments, cells comprising the TRAIL-R2-targeting chimeric protein expression construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### VII. Cells

The present disclosure encompasses cells of any kind that harbor a vector that encodes the TRAIL-R2-targeting chimeric protein and that optionally may encode one or more heterologous proteins, such as a CAR or a transgenic TCR. The cells may be immune cells, including conventional T cells, NK cells, gamma-delta T cells, NKT and invariant NK T cells, regulatory T cells, macrophages, B cells, tumor infiltrating lymphocytes, or a mixture or derivatives thereof may be employed, but in particular cases the cells are peripheral blood-activated T cells. The cells may be any kind of stem cells, as well, including mesenchymal stem cells.

Following genetic modification with the vector(s), the cells may be immediately infused or may be stored. In certain aspects, following genetic modification, the cells may be propagated for days, weeks, or months ex vivo as a bulk population within about 1, 2, 3, 4, 5 days or more following gene transfer into the cells. In a further aspect, the transfectants are cloned and a clone demonstrating presence of a single integrated or episomally maintained expression cassette or plasmid, and expression of the chimeric protein is expanded ex vivo. The clone selected for expansion may be shown to demonstrate the capacity to specifically recognize TRAIL-R2-expressing target cells. In certain cases, the immune cells may be expanded by stimulation with one or more cytokines. The recombinant immune cells may be expanded by stimulation with artificial antigen presenting cells. In a further aspect, the genetically modified cells may be cryopreserved.

Certain embodiments of the disclosure encompass cells that express the TRAIL-R2 chimeric protein, one or more CARs, and optionally one or more other heterologous proteins as encompassed herein. The cell comprises a recombinant nucleic acid that encodes the TRAIL-R2-targeting chimeric protein and one or more CARs and optionally a suicide gene, in specific embodiments.

The cells may be obtained from an individual directly or may be obtained from a depository or other storage facility. The cells as therapy may be autologous or allogeneic with respect to the individual to which the cells are provided as therapy.

The cells may be from an individual in need of therapy for a medical condition, and following their manipulation to express the TRAIL-R2-targeting chimeric protein, they may be provided back to the individual from which they were originally sourced. In some cases, the cells are stored for later use for the individual or another individual. In such cases, the cells may or may not be further modified prior to use.

The cells that harbor the TRAIL-R2-targeting chimeric protein may be comprised in a population of cells, and that population may have a majority that are transduced with a polynucleotide that encodes the TRAIL-R2-targeting chimeric protein. A cell population may comprise 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of cells that are transduced with a polynucleotide that encodes the TRAIL-R2-targeting chimeric protein.

The cells may be produced with the TRAIL-R2-targeting chimeric protein for the intent of being modular with respect to a specific purpose. For example, cells may be generated, including for commercial distribution, expressing a TRAIL-R2-targeting chimeric protein and a user may modify them to express one or more other genes of interest (including therapeutic genes) dependent upon their intended purpose(s). For instance, a party interested in treating a certain antigen-positive cancer may modify them to express a CAR for the antigen. Other modifications to the cells may include modifications that tailor the cells to the needs of the individual receiving the cells, including a need specific for a particular type of cancer with which the individual is afflicted.

### VIII. Methods of Treatment

In various embodiments TRAIL-R2-targeting chimeric protein constructs, nucleic acid sequences, vectors, host cells and so forth as contemplated herein and/or pharmaceutical compositions comprising the same are used for the prevention, treatment or amelioration of a cancerous disease, such as a tumorous disease. In particular embodiments, the pharmaceutical composition of the present disclosure may be particularly useful in preventing, ameliorating and/or treating cancer, including cancers that may or may not be solid tumors, for example. The individual may utilize the treatment method of the disclosure as an initial treatment or after (or with) another treatment, for example. The immunotherapy methods may be tailored to the need of an individual with cancer based on the type and/or stage of cancer, and in at least some cases the immunotherapy may be modified during the course of treatment for the individual.

In some embodiments, the present disclosure provides methods for immunotherapy comprising administering an effective amount of the cells produced by methods of the present disclosure. In one embodiment, a medical disease or disorder is treated by transfer of cell populations produced by methods herein and that elicit an immune response. In certain embodiments of the present disclosure, cancer is treated by transfer of a cell population produced by methods of the disclosure and that elicits an immune response. Provided herein are methods for treating or delaying progression of cancer in an individual comprising administering to the individual an effective amount TRAIL-R2-targeting cell therapy. The present methods may be applied for the treatment of solid cancers or hematologic cancers.

Tumors for which the present treatment methods are useful include any malignant cell type, such as those found in a solid tumor or a hematological tumor. Exemplary solid tumors can include, but are not limited to, a tumor of an organ selected from the group consisting of brain cancer, lung cancer, renal cancer, liver cancer, prostate cancer, thyroid cancer, stomach cancer, ovarian cancer, endometrial cancer, bladder cancer, melanoma, breast cancer, head and neck cancer, mesothelioma, bone cancer, colon cancer, esophageal cancer, gall bladder cancer, appendicular cancer, cervical cancer, cholangiocarcinoma, pancreatic cancer, sarcomas, and so forth. Hematological cancers include at least acute myeloid leukemia, lymphoma, multiple myeloma, and so forth

In certain embodiments of the present disclosure, TRAIL-R2-targeting immune cells are delivered to an individual in need thereof, such as an individual that has cancer. The cells then enhance the individual's immune system to attack the cancer cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, or more days.

In specific embodiments, the cells that have been engineered to express TRAIL-R2-targeting chimeric protein are provided to an individual in a therapeutically effective amount (in a range from 10³ to 10¹⁰) that ameliorates at least one symptom related to cancer cells in the individual. A therapeutically effective amount may be from 10³ to 10¹⁰, 10³ to 10⁹, 10³ to 10⁸, 10³ to 10⁷, 10³ to 10⁶, 10³ to 10⁵, 10³ to 10⁴, 10⁴ to 10¹⁰, 10⁴ to 10⁹, 10⁴ to 10⁸, 10⁴ to 10⁷, 10⁴ to 10⁶, 10⁴ to 10⁵, 10⁵ to 10¹⁰, 10⁵ to 10⁹, 10⁵ to 10⁸, 10⁵ to 10⁷, 10⁵ to 10⁶, 10⁶ to 10¹⁰, 10⁶ to 10⁹, 10⁶ to 10⁸, 10⁶ to 10⁷, 10⁷ to 10¹⁰, 10⁷ to 10⁹, 10⁷ to 10⁸, 10 to 10¹⁰, 10 to 10⁹, or 10⁹ to 10¹⁰ cells. Thus, in particular embodiments an individual having a certain cancer is provided once or multiple times a therapeutically effective amount of cells expressing TRAIL-R2-targeting chimeric proteins.

In particular embodiments, the present disclosure contemplates, in part, TRAIL-R2-targeting chimeric protein-expressing cells, TRAIL-R2-targeting chimeric protein constructs, TRAIL-R2-targeting chimeric protein nucleic acid molecules and TRAIL-R2-targeting chimeric protein vectors that can administered either alone or in any combination using standard vectors and/or gene delivery systems, and in at least some aspects, together with a pharmaceutically acceptable carrier or excipient. In certain embodiments, subsequent to administration, the nucleic acid molecules or vectors may be stably integrated into the genome of the subject.

In specific embodiments, viral vectors may be used that are specific for certain cells or tissues and persist in immune cells, for example. Suitable pharmaceutical carriers and excipients are well known in the art. The compositions prepared according to the disclosure can be used for the prevention or treatment or delaying cancer.

Furthermore, the disclosure relates to a method for the prevention, treatment or amelioration of a tumorous disease comprising the step of administering to a subject in the need thereof an effective amount of cells that express a TRAIL-R2-targeting chimeric protein, a nucleic acid sequence, a vector, as contemplated herein and/or produced by a process as contemplated herein.

The administration of the composition(s) of the disclosure is useful for all stages and types of cancer, including for minimal residual disease, early cancer, advanced cancer, and/or metastatic cancer and/or refractory cancer, for example.

Therapeutically effective amounts of the produced cells can be administered by a number of routes, including parenteral administration, for example, intravenous, intraperitoneal, intramuscular, intrasternal, intratumoral, intrathecal, intraventricular, through a reservoir, intraarticular injection, or infusion.

The therapeutically effective amount of the produced cells for use in adoptive cell therapy is that amount that achieves a desired effect in a subject being treated. For instance, this can be the amount of immune cells necessary to inhibit advancement, or to cause regression of cancer.

The produced cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of cells will be dependent on the subject being treated, the severity and type of the affliction, and the manner of administration. In some embodiments, doses that could be used in the treatment of human subjects range from at least 1×10³, at least 1×10⁴, 3.8×10⁴, at least 3.8×10⁵, at least 3.8×10⁶, at least 3.8×10⁷, at least 3.8×10⁸, at least 3.8×10⁹, or at least 3.8×10¹⁰ T cells/m². In a certain embodiment, the dose used in the treatment of human subjects ranges from about 3.8×10⁹ to about 3.8×10¹⁰ T cells/m². In additional embodiments, a therapeutically effective amount of T cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of T cells is readily determined by one of skill in the art based on the age, weight, sex, and physiological condition of the subject. Effective doses can be extrapolated from dose-response curves derived from in vitro or animal model test systems.

The disclosure further encompasses co-administration protocols with other compounds, e.g. bispecific antibody constructs, targeted toxins or other compounds, which act via immune cells. The clinical regimen for co-administration of the inventive compound(s) may encompass co-administration at the same time, before or after the administration of the other component. Particular combination therapies include chemotherapy, radiation, surgery, hormone therapy, or other types of immunotherapy.

Embodiments relate to a kit comprising a TRAIL-R2-targeting chimeric protein construct as defined herein, a nucleic acid sequence as defined herein, a vector as defined herein and/or a host as defined herein. It is also contemplated that the kit of this disclosure comprises a pharmaceutical composition as described herein above, either alone or in combination with further medicaments to be administered to an individual in need of medical treatment or intervention.

### IX. Kits of the Disclosure

Any of the compositions described herein may be comprised in a kit. In a non-limiting example, the kit comprises TRAIL-R2-targeting chimeric protein molecules, cells encompassing same, and/or reagents to generate same, and any of these may be comprised in suitable container means in a kit of the present disclosure. Kits may comprise immune cells, including T cells, vectors, expression construct polynucleotides for insertion into a vector (whether viral or not), particular sequences of any kind and as encompassed herein, and so forth. Primers for amplification of any polynucleotide may be included. In some cases, the kit comprises cryopreserved cells, including T cells whether or not they comprise vectors that encode the TRAIL-R2-targeting chimeric protein. Any reagents for transfection or transduction of the cells may be included.

The compositions of the kits may be packaged either in aqueous media or in lyophilized form. The container means of the kits will generally include at least one vial, test tube, flask, bottle, syringe or other container means, into which one or more components may be placed, and preferably, suitably aliquoted. Where there are more than one component in the kit, the kit also may generally contain a second, third or other additional container into which the additional components may be separately placed. However, various combinations of components may be comprised in a vial. The kits of the present disclosure also will typically include a means for containing the TRAIL-R2-targeting chimeric protein molecules, cells encompassing same, and/or reagents to generate same in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly envisioned. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

Irrespective of the number and/or type of containers, the kits of the disclosure may also comprise, and/or be packaged with, an instrument for assisting with the injection/administration and/or placement of the ultimate composition within the body of an animal. Such an instrument may be a syringe, pipette, forceps, and/or any such medically approved delivery vehicle. In some embodiments, reagents or apparatuses or containers are included in the kit for ex vivo use.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Overcoming the Tumor Microenvironment for Cart Cell Therapy by Targeting Myeloid Derived Suppressor Cells (MDSCS) Through a Trail-R2-Specific Receptor

There have been extraordinary successes with CART cell therapy for hematological malignancies. Broadening the application of CART cells to include solid tumors as targets, however, has proven more challenging. One of the main obstacles to overcome for this therapy to be successful in solid tumors is the hostile tumor microenvironment (TME). The TME is composed of cells and secreted cytokines that are potently immunosuppressive and block the activity of CART cells. In many solid tumors, myeloid derived suppressor cells (MDSCs) are the main contributors to induction and maintenance of T cell suppression. The accumulation of MDSCs at the tumor site has been associated with poor prognosis, increased disease burden and importantly, poor response to immunotherapies. Armed with these facts, investigators have focused on finding ways to eliminate MDSCs from the TME. Most strategies are nonspecific and have significant side effects.

However, a targeted approach is to use an agonist mAb against TNF-related apoptosis induced ligand receptor 2 (TRAIL-R2); a receptor expressed on MDSCs that activates apoptosis upon engagement with its soluble ligands. The present disclosure concerns the engineering of a receptor that engages with TRAIL-R2, therefore inducing apoptosis of MDSCs and at the same time provides a costimulatory signal to CART cells that improve their persistence and antitumor effects. For this receptor the inventors used the scFv of a monoclonal antibody specific for TRAIL-R2 as the extracellular domain of the receptor and as signaling the intracellular domain of 4-1BB. This receptor is referred to as TR24-1BB. T cells engineered to express TR24-1BB are able to specifically eliminate MDSCs in vitro and in vivo show improved antitumor effects as well as higher proliferation and persistence at the tumor site compared to CART cells alone.

### Example 2

### Overcoming the Breast Tumor Microenvironment by Targeting MDSCS Through Car-T Cell Therapy

Broadening the application of CAR T cells (CARTc) to include solid tumors has proven challenging, largely due to the hostile immune suppressive tumor microenvironment (TME). In breast cancer (BC), myeloid derived suppressor cells (MDSCs) are the main contributors to the induction and maintenance of T cell inhibition within the TME. To effectively target BC we developed and validated a CAR targeting tumor-expressed mucin 1 (MUC1) (Bajgain P et al, 2018). In order to potentiate the expansion and persistence of MUC1 CARTs and regulate the TME, a novel construct was generated encoding a chimeric receptor called TR2.4-1BB, encoding a TNF-related apoptosis-inducing ligand receptor 2 (TR2) specific scFv linked with 4-1BB endodomain. It was considered that upon exposure to TME-resident MDSCs, which express high levels of TR2, CAR T cells receive an activating co-stimulatory signal which will promote T cell persistence and expansion. To evaluate the benefit of co-expression of both receptors rendering CAR T cells resistant to the TME, the phenotypic profile, proliferative capacity, cytotoxic activity and anti-tumor effects were compared of T cells expressing either CAR.MUC1 alone, TR2.4-1BB alone or a combination of both MUC1.TR2.4-1BB CARTc. T cells were activated with OKT3/CD28 antibodies and then transduced with retroviral supernatants, resulting in high levels of expression of each transgenic receptor (87.3±4%, 80.8±2.7% and 74±5.5% transduction efficiency for MUC1, TR2.4-1BB and MUC1.TR2.4-1BB CARTc, respectively). To confirm that the chimeric receptor TR2.4-1BB was functional, transgenic cells were exposed to recombinant TR2 and evaluated NFKB translocation (due to 41BB signaling), which was induced only in cells that expressed TR2.4-1BB (percent absorbance: 0±0% and 3±0% NFkB translocation, NT and TR2.4-1BB respectively). Co-culture of OKT3/CD28 stimulated non-transduced T cells with MDSCs significantly decreased T cell proliferation by 75±5% and IFN-γ production by half. Moreover, in presence of MDSCs, cytotoxic activity of MUC1 CARTc is diminished by 25% against MUC1+BC cell lines in a 4 hr cytotoxicity assay. However, transgenic TR2.4-1BB expression induced MDSC apoptosis in a co-culture cytotoxicity assay (70±7% vs 15±2% CAR.MUC1 alone). Finally, to assess the anti-tumor effects associated with co-expression of CAR.MUC1 and TR2.4-1BB on T cells, a xenograft model was established whereby mice were co-injected with BC tumors with or without MDSCs in the mouse mammary fat pad. Addition of MDSCs led to increased tumor growth (almost two-fold) and distant metastatic spread compared to tumor cells alone. Treatment with Firefly Luciferase-labeled MUC1.TR2.4-1BB CARTc resulted in a significant delay in tumor growth (576.5±87.8 mm³) compared with CAR.MUC1 (1131.9±89.2 mm³) or TR2.4-1BB (1032.6±46.4 mm³) T cells alone and improved T cell proliferation and persistence at the tumor site (2.14×10⁸±7.91×10⁷p/s vs 8.02×10⁷±7.88×10⁷p/s and 1.06×10⁷±9.10×10⁷p/s for CAR.MUC1 and TR2.4-1BB respectively). In conclusion, the data indicate that CARTc co-expressing TR2.4-1BB receptor have higher anti-tumor potential against BC tumors by inducing apoptosis of MDSCs, therefore remodeling the TME and improving T cell proliferation at the tumor site.

### Example 3

### Selectively Targeting Myeloid Derived Suppressor Cells (MDSCS) Through Trail Receptor 2 (TR2) to Enhance the Efficacy of Car T Cell Therapy for the Treatment of Breast Cancer

Background: Successful targeting of solid tumors such as breast cancer (BC) using CAR T cells (CARTs) has proven challenging, largely due to the immune suppressive tumor microenvironment (TME). Myeloid derived suppressor cells (MDSCs) inhibit CART's function and persistence within the breast TME. CAR T cells were generated targeting tumor-expressed mucin 1 (MUC1) (Bajgain P et al, 2018) for BC. To potentiate expansion and persistence of MUC1 CARTs and modulate the suppressive TME, a novel chimeric co-stimulatory receptor was generated, TR2.4-1BB, encoding a ScFv derived from a TNF-related apoptosis-inducing ligand receptor 2 (TR2) mAb followed by a 4-1BB endodomain. It was considered that engagement with TR2 expressed on TME-resident MDSCs, will lead to both MDSC apoptosis and CART co-stimulation, promoting T cell persistence and expansion at tumor site.

Methods: Function of the novel TR2.4-1BB receptor, was assessed by exposing non-transduced (NT) and TR2.4-1BB transduced T cells to recombinant TR2 and nuclear translocation of NFkB was measured by ELISA. Functionality of in vitro generated MDSCs was determined by the suppression assay. In vitro CART/co-stimulatory receptor T cell function was measured by cytotoxicity assays using MUC1+ tumor targets in presence or absence of MDSCs. In vivo anti-tumor activity was assessed using MDSC enriched tumor-bearing mice using calipers to assess tumor volume and bioluminescence imaging to track T cells.

Results: Nuclear translocation of NFkB was detected only in TR2.4-1BB T cells. MDSCs significantly attenuated T cell proliferation by 50±5% and IFNg production by half compared with T cells cultured alone. Additionally, presence of MDSCs, diminished cytotoxic potential of MUC1 CARTs against MUC1+BC cell lines by 25%. However, TR2.4-1BB expression on CAR.MUC1 T cells induced MDSC apoptosis thereby restoring the cytotoxic activity of CAR.MUC1 against MUC1+BC lines in presence of TR2.4-1BB (67±8.5%). There was an approximate two-fold increase in tumor growth due enhanced angiogenesis and fibroblast accumulation in mice receiving tumors+MDSCs compared to tumors alone. Treatment of these MDSC-enriched tumors with MUC1.TR2.4-1BB CARTs led to superior tumor cell killing and significant reduction in tumor growth (24.54±8.55 mm³) compared to CAR.MUC1 (469.79.9±81.46 mm³) or TR2.4-1BB (434.86±64.25 mm³) T cells alone (Day 28 after T cell injection). The treatment also improved T cell proliferation and persistence at the tumor site. Thereby, leading to negligible metastasis demonstrating ability of CARTs to eliminate tumor and prevent dissemination. Similar results were observed using HER2.TR2.4-1BB CARTs in a HER2+BC model.

Conclusions: The findings demonstrate that CARTs co-expressing the novel TR2.4-1BB receptor have higher anti-tumor potential against BC tumors and infiltrating MDSCs, resulting in TME remodeling and improved T cell proliferation at the tumor site.

### Example 4

### Examples of Materials and Methods

Donor and Cell Lines

Peripheral blood mononuclear cells (PBMCs) were obtained from healthy volunteers from Gulf Coast regional blood center, after informed consent on protocols approved by the Baylor College of Medicine Institutional Review Board. The 293T and human breast cancer cell lines MDA-MB-231, SUM-159, MDA-MB-453 and BT-20 were obtained from the American Type Culture Collection (ATCC) and were grown in Dulbecco's Modified Eagle Medium (DMEM, GE Healthcare Life Sciences) supplemented with 10% FBS and 2Mm L-Glutamax. All cell lines were maintained in a humidified atmosphere containing 5% carbon dioxide (CO₂) at 37° C.

Generation of Retroviral Constructs and Retroviral Supernatant

A codon-optimized 2^{nd} generation CAR was used with specificity against tumor associated MUC1²⁵. This CAR uses a HMFG2 scFv coupled to a IgG2-CH3 spacer domain followed by a CD28 transmembrane domain the CD28 co-stimulatory endodomain and CD3 (domain were all cloned in a SFG retroviral backbone. To generate the novel co-stimulatory receptor an SGF-based retroviral vector was engineered to encode a scFv derived from a TRAIL-R2 mAb²⁴ fused to IgG2-CH3 spacer domain followed by a 4-1BB co-stimulatory endodomain. The HER2-specific CAR was constructed by subcloning the HER2-specific single chain variable fragment FRP5 into an SFG retroviral vector containing a CD28 transmembrane and a CD28.ζ signaling domain²⁶. Retroviral supernatant for both the CARs and co-stimulatory receptor was produced using 293T cells, which were co-transfected with 3.75 mg of CAR.MUC1, TRAIL-R2.4-1BB or CAR.HER2 retroviral vector, 3.75 mg of Peg-Pam-e plasmid containing the sequence for the MoMLV gag-pol, and 2.5 μg of the RDF plasmid containing the sequence for the RD114 envelope, using the GeneJuice transfection reagent (Novagen). Retroviral supernatant was collected at 48 and 72 hrs post-transfection, filtered using a 0.45-mm filter, and stored at −80° C.

Generation of CAR T Cells

The CD14⁻ PBMC fraction was used to generate CAR T cells. 1×10⁶ PBMCs were plated in each well of a non-tissue culture treated 24-well plate that was pre-coated with CD3 (1 mg/ml) (Ortho Biotech, Inc., Bridgewater, N.J.) and CD28 (1 mg/ml) (Becton Dickinson & Co., Mountain View, Calif.). The cells were cultured in complete CTL media (RPMI-1640 containing 45% Clicks medium, 10% FBS and 2Mm L-Glutamax) supplemented with recombinant human IL-2 (50 U/mL) on Day 1. On day 3 post CD3/CD28 T cell generation, 1 mL of retroviral supernatant was added to non-tissue culture treated 24-well plate pre-coated with recombinant fibronectin (FN CH-296; Retronectin; Takara Shuzo, Otsu, Japan) and centrifuged at 2000 g for 90 minutes. CD3/CD28 activated T cells (0.25×10⁶ cells/mL) were resuspended in complete CTL media supplemented with IL-2 (100 U/mL) and then added to the wells and centrifuged at 400 g for 5 minutes. To generate CAR.MUC1 and TRAIL-R2.4-1BB co-expressing cells, activated T cells were transduced²⁵ sequentially first with CAR.MUC1 and then with TRAIL-R2.4-1BB on days 3 and 4 respectively. Transduction efficiency was measured 3 days post flow cytometry. CAR.HER2 and TRAIL-R2.4-1BB co-expressing cells were generated similarly.

Generation of M-MDSCs

PBMCs were obtained from healthy volunteers after informed consent on protocols approved by the Baylor College of Medicine Institutional Review Board. CD14+ cells were isolated using anti-CD14 magnetic microbeads and LS column separation (Miltenyi Biotech) per manufacturer's instructions. These cells were then cultured in complete RPMI media (10% FBS and 2Mm L-Glutamax) for 7 days in presence of GM-CSF and IL-6. GM-CSF was added on day 1, 3 and 5, whereas IL-6 was added only on day 5. The cells were then harvested a week later, and flow-cytometry was performed to assess the phenotype.

ELISA

NT or TRAIL-R2.4-1BB transduced T cells were cultured alone (negative control) or in presence of recombinant TRAIL-R2 protein or anti-CD3 and anti-CD28 (positive control). The cells were then harvested at 0, 60 and 120 minutes and the nuclear fraction was collected. An ELISA for NFkB was performed using the NFkB p65 Transcription Factor Assay Kit (ab133112; Abcam), following manufacturer's instructions. Absorbance was measured at 450 nm with a TECAN Infinite M200 Pro plate reader.

Flow Cytometry

MUC1 antigen expression by tumor cells was measured using MUC1-AF700 (clone SM3/Cat #NB12022711) and HER2 expression was measured using HER2-APC (clone 24D2/Cat #324408). MUC1.CAR was detected using anti-CH2 spacer antibody conjugated with AF647 and HER2.CAR was detected using the recombinant HER2/Fc chimera (R&D Systems Cat #1129-ER-050) followed by addition of the goat anti-human IgG Fc-AF647 secondary antibody (SouthernBiotech Cat #2014-31). The novel TRAIL-R2.4-1BB receptor was detected using biotinylated TRAIL-R2 protein (Cat #TR2-H82E6) followed by addition of Streptavidin-BV421 (Cat #405226). The following antibodies were used for MDSC phenotyping: CD33-PE (clone WM53/Cat #555450), FITC-HLA-DR (clone G46-6/Cat #556643), CD11b-APC (clone ICRF44/Cat #301310), CD14-APC.Cy7 (clone HCD14/Cat #325620), CD15-BV510 (clone W6DE/Cat #323028), TRAIL-R2-BV421 (clone YM366/Cat #743835). Surface expression of TRAIL-R2 on T, B and NK cells was assessed using the following panel: CD45-AF700 (clone J33/Cat #A71117), CD3-PE (clone SK7/Cat #347347), CD4-Krome orange (clone 13B8.2/Cat #A96417), CD8-PC7 (clone SFCI21Thy2D3/Cat #6607102), FITC-CD56 (clone NCAM16.2/Cat #340410), CD19-PerCP (clone 4G7/Cat #347544) and TRAIL-R2-BV421 (clone YM366/Cat #743835). T cell phenotyping was carried out using 3 different panels, the memory marker panel included the following antibodies: CD3 AF-AF750 (clone UCHT1/Cat #A66329), CD4-PE (clone SK3/Cat #347327), CD8-PerCP (clone SK1/Cat #347314), CD62L-APC (clone DREG-56/Cat #559772), CD45RA-PacBlue (clone 2H4/Cat #A82946) and CD3 AF-AF750 (clone UCHT1/Cat #A66329). The exhaustion panel consisted of the following antibodies: CD3 AF-AF750 (clone UCHT1/Cat #A66329), CD4-Krome orange (clone 13B8.2/Cat #A96417), CD8-PerCP (clone SK1/Cat #347314), PD1-PE (clone MIH4/Cat #557946), TIM3-APC (clone F38-E2E/Cat #345011) and 4-1BB-BV421 (clone 4B4-1/Cat #564091). The activation panel included antibodies: CD3 AF-AF750 (clone UCHT1/Cat #A66329), CD4-Krome orange (clone 13B8.2/Cat #A96417), CD8-AF700 (clone B9.11/Cat #A66332), PE-CD25 (clone M-A251/Cat #555432) and CD69-PerCP (clone L78/Cat #340548). Cells were stained with (˜2 uL) of antibody for 20 mins at 4° C., washed (PBS, Sigma-Aldrich) and then acquired on Gallios™ Flow Cytometer (Beckman Coulter Inc.). Analysis was performed using Kaluza Flow Analysis Software (Beckman Coulter Inc.).

Determination of Apoptosis

PBMCs (resting or activated) were labelled with CFSE and co-cultured in the presence of non-transduced (NT) T cells, transduced CAR.MUC1, TRAIL-R2.4-1BB or CAR.MUC1.TRAIL-R2.4-1BB T cells. Cells were harvested at 6 hr and 24 hr, apoptosis was evaluated using Annexin-V-PE apoptosis detection kit with 7-AAD (Cat #640934) by flow cytometry. Cells were washed twice with cold PBS and re-suspended in Annexin V binding buffer. They were then stained with 5 mL of Annexin-V-PE and 5 mL of 7-AAD viability staining solution for 15 mins at room temperature in the dark prior to flow cytometric analysis.

Chromium-Release Assay

The cytotoxic specificity of effector T cells was measured in a standard 4- or 6-hr 51 chromium (⁵¹Cr) release assay using E:T ratios of 40:1, 20:1, 10:1, and 5:1. Effector T cells (NT, CAR.MUC1, TRAIL-R2.4-1BB, CAR.HER2, CAR.MUC1.TRAIL-R2.4-1BB and CAR.HER2.TRAIL-R2.4-1BB) were co-incubated in triplicates with target cells labeled with ⁵¹Cr in a V-bottomed 96-well plate. Targets included BT-20, MDA-MB-231, MDA-MB-453 and MDSCs (5-6-hr assay). At the end of their respective incubation periods at 37° C. and 5% CO₂, supernatants were harvested, and radioactivity was counted in a gamma counter.

Percentage of specific lysis was calculated as follows: % specific cytotoxicity=[experimental release (cpm)−spontaneous release (cpm)]/[maximum release (cpm)−spontaneous release (cpm)]×100.

Suppression Assay

The suppressive function of MDSCs was measured by their ability to inhibit the proliferation of autologous T cells. Fresh T cells were isolated from PBMCs of autologous donors by anti-CD3 microbeads and magnetic LS column separation (Miltenyi Biotech), they were labeled with 1 μM 5, 6-carboxyfluorescein diacetate, succinimidyl ester (CFSE) and seeded in a 24-well plate. Autologous MDSCs were added to these wells at a 1:1 or 1:2 ratio. T cells were stimulated with anti-CD3/CD28 mAbs (Invitrogen) and IL-2 (200 U/mL, R & D Systems) and then cultured alone or with MDSCs. After cultured for 3 days, cells were harvested and stained with CD4-PacBlue (clone 13B8.2/Cat #A82789) and CD8-APC (clone B9.11/Cat #IM2469 U) antibodies. T cell proliferation was determined by flow cytometric analysis.

Immunohistochemistry

Paraffin-embedded tumor tissues from tumor and tumor+MDSC treated mice were sectioned. The sections (5 μm) were deparaffinized in xylene, dehydrated and rehydrated in ethanol, and subjected to steamer antigen retrieval. The sections were then processed with the following antibodies: mouse anti-CD31 and human anti-aSMA. A total of 10-14 fields of view were analyzed for each group.

Western Blot Assay

Resting, activated T cells and MDSCs were washed three times with ice-cold PBS. Protein lysate was extracted by RIPA lysis buffer containing protease inhibitor cocktail for 20 mins on ice, followed by centrifugation at 12,500 g for 20 mins at 4° C. The protein concentration was determined using the BCA protein assay kit (Thermo Fisher Scientific). Equivalent amounts of protein samples were separated by sodium dodecyl sulfate-polyacrylamide gel electrophoresis (SDS-PAGE) gels, and then transferred to a polyvinylidene difluoride (PVDF) membrane (Bio-Rad). After blocking with 5% (w/v) nonfat milk overnight at 4° C., the blots were incubated with primary antibodies (anti-cFLIP (clone 7F10/Cat #ALX-804-961-0100) and anti-GAPDH (clone 6C5/Cat #sc-32233)) overnight at 4° C. After washing for three times, the membranes were further incubated with corresponding secondary antibody (IRDYE 680RD goat anti-mouse) for 2 hr at room temperature. The membrane was imaged on Licor, and the cFLIP expression was normalized to the internal control, GAPDH.

In Vivo Study

For the in vivo cell derived xenograft (CDX) mouse model, six to eight-week-old female NSG mice (NOD.CgPrkdcscid Il2rgtm1Wjl/SzJ, Jackson ImmunoResearch Laboratories) were injected with 5×10⁶ GFP.ffLuc.SUM-159 cells alone and/or 5×10⁶ MDSCs suspended in 50% DPBS/50% matrigel subcutaneously (s.c.) into the left 4th mammary fat pad. Tumor burden was monitored using the IVIS Lumina In Vivo Imaging system (Caliper Life Sciences, Hopkinton, Mass.) 15 mins after injection (i.p.) with 100 ul of D-luciferin (15 mg/mL). Tumor size was measured by bi-weekly caliper measurement and tumor volume (mm³) was calculated by length×width×width/2. For the in vivo CARTc therapy study, the above xenograft mouse model was used and once the tumors reached a size of approximately 100 mm3 (˜ 2 weeks), animals were injected on day 14 intravenously (i.v.) with 1.5×10⁶ CAR.MUC1, TR2.41BB, CAR.MUC1.TR2.41BB T cells. Tumor size was measured by bi-weekly caliper measurement and tumor volume (mm³) was calculated by length×width×width/2. For the MDA-MB-231 CDX mouse model, NSG mice were implanted with 5×10⁶ MDA-MB-231 cells alone and/or 5×10⁶ MDSCs. Once tumors reached approximately 100 mm³ (˜2 weeks), animals were injected on day 14 intravenously (i.v.) with 1.5×10⁶ GFP.ffLuc+CAR.MUC1, TR2.41BB, CAR.MUC1.TR2.41BB T cells. T cell expansion and persistence was monitored using the IVIS Lumina In Vivo Imaging system (Caliper Life Sciences, Hopkinton, Mass.) 15 mins after injection (i.p.) with 100 ul of D-luciferin (15 mg/mL). All in vivo analysis was performed using Living Image software (Caliper Life Sciences, Inc., Hopkinton, Mass.). For the MDA-MB-453 HER2+ CDX mouse model, MDA-MB-453 cells were subcutaneously (s.c.) injected into the left 4th mammary fat pad of NSG mice. On day 14 MDSCs were administered to some of these mice and on day 21 mice were i.v. injected with 1.5×10⁶ GFP.ffLuc+CAR.HER2, TR2.41BB, CAR.HER2.TR2.41BB T cells. All the parameters were measured similarly as stated above.

Statistical Analysis

All statistical analysis was performed using the GraphPad Prism 8.01 statistical software. The results were expressed as the mean of arbitrary values±standard error mean (SEM). Statistical significance between groups was assessed by two-way ANOVA followed by Tukey's multiple comparison, or unpaired two tailed t-test where a p-value less than 0.05 denoted statistical significance.

### Example 5

### The Novel Trail-R2.4-1Bb Co-Stimulatory Receptor Specifically Targets Trail-R2 and Induces Downstream Signaling

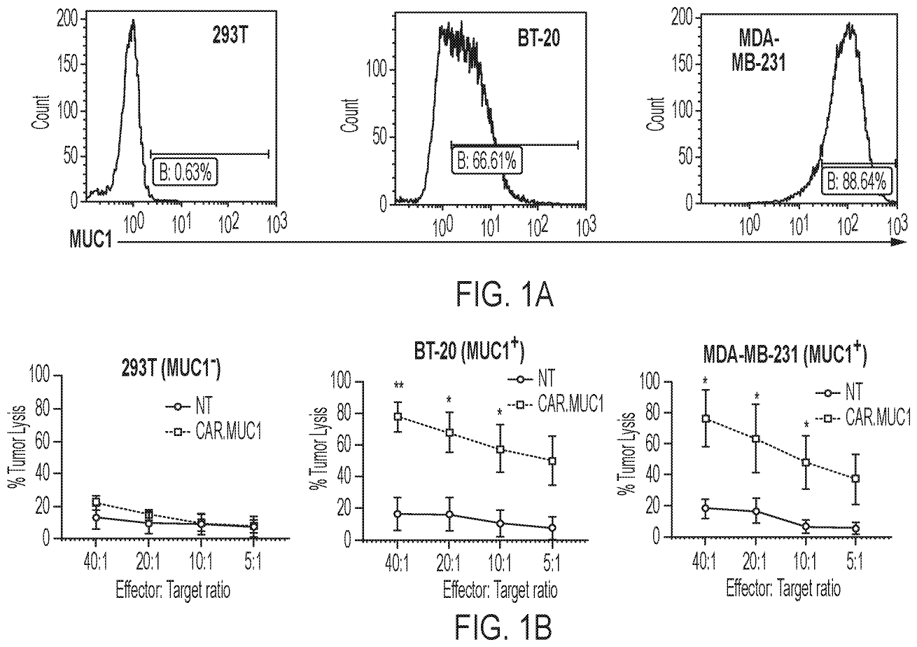

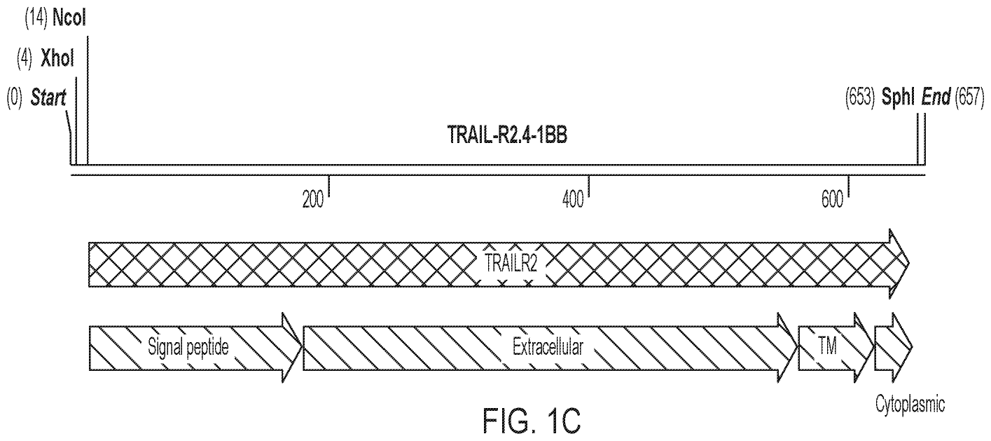

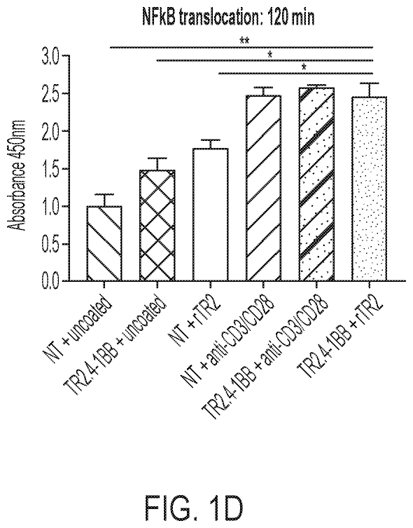

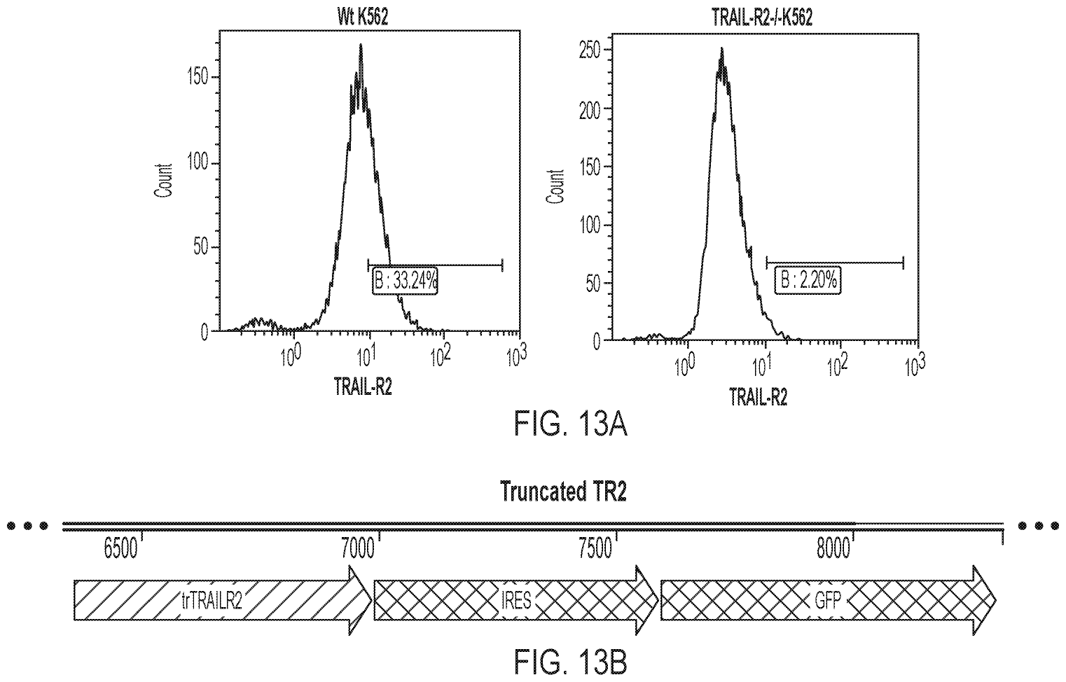

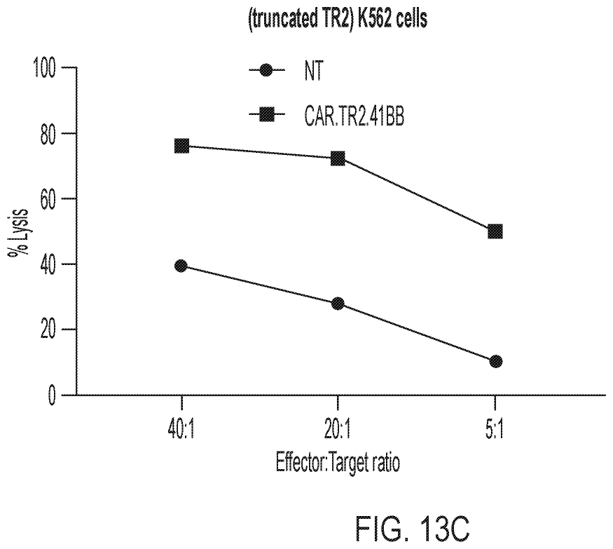

To specifically target TNBC a previously generated and validated retroviral vector was used encoding a 2^{nd} generation human, codon optimized CAR directed against the tumor associated antigen MUC1²⁵. It was first confirmed that CAR.MUC1 transduced T cells are able to specifically eliminate MUC1 expressing breast cancer cell lines (BT-20 and MDA-MB-231) with no killing of MUC1 negative 293T cell line (FIGS. 1A and 1B). To overcome the tumor microenvironment (TME), a novel co-stimulatory receptor was generated that targets TRAIL-R2, with the purpose of inducing MDSC apoptosis. To confirm specific binding and signaling of the TRAIL-R2 receptor, an SFG-based retroviral CAR vector was engineered encoding the scFv derived from a TRAIL-R2 mAb²⁴ followed by a 4-1BB signaling domain and the CD3z domain. In order to differentiate between specific killing through the TRAIL-R2 CAR vs TRAIL-R2 death receptor signaling on the target cells, TRAIL-R2 was knocked out on K562 cells and then transduced with a truncated version of TRAIL-R2, lacking the functional death domain (FIGS. 13A-13B). The TRAIL-R2.4-1BB.CD3z transduced T cells were able to specifically target truncated TRAIL-R2+K562 cells (75% lysis at 40:1 effector to target ratio) (FIG. 13C). Having established the specificity of the TRAIL-R2.4-1BB. CD3z receptor, it was determined whether the co-stimulatory receptor alone in absence of the CD3z was able to trigger downstream signaling through the 4-1BB domain. A finalized co-stimulatory receptor was generated by encoding the same TRAIL-R2 mAb scFv followed by a 4-1BB signaling domain (FIG. 1C). Using ELISA assay, translocation was confirmed of NFkB into the nucleus following engagement of the receptor with recombinant TR2 as a read-out of downstream signaling through the 4-1BB domain. The ligation of TRAIL-R2.4-1BB receptor with recombinant TRAIL-R2 resulted in translocation of NFkB into the T cell nucleus at levels similar to what was detected in presence of anti-CD3 and anti-CD28 (percent absorbance: 0±0% and 3±0% NFkB translocation, NT and TRAIL-R2.4-1BB respectively, mean±SEM, n=3) (FIG. 1D). This data shows that the TRAIL-R2.4-1BB construct demonstrates specificity and is able to induce downstream T cell co-stimulatory signaling through the 4-1BB domain.

### Example 6

### Myeloid Derived Suppressor Cells (MDSCS) Generated from Peripheral Blood Suppress T Cell Function and Proliferation

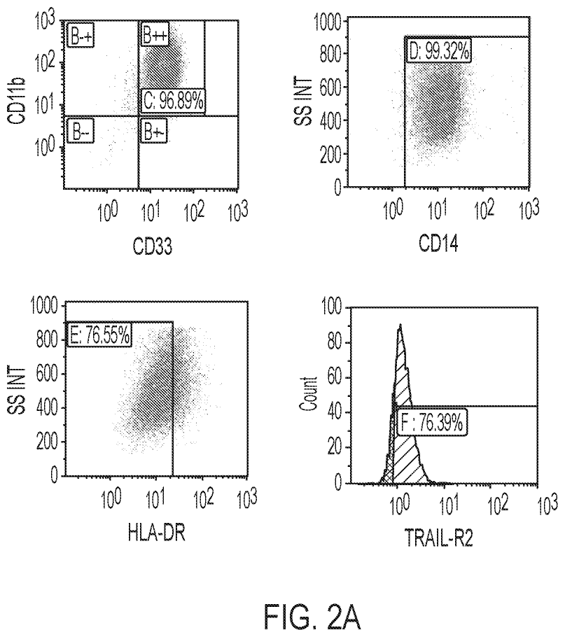

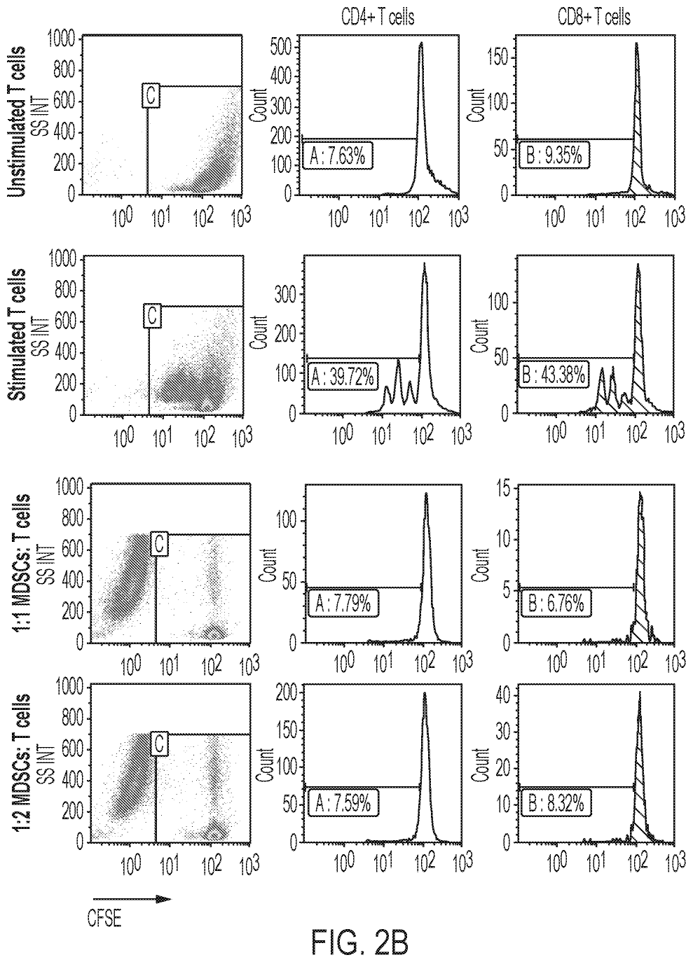

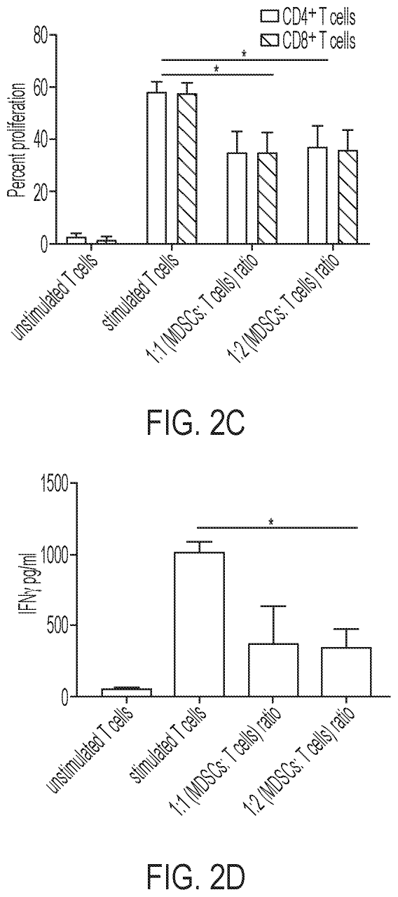

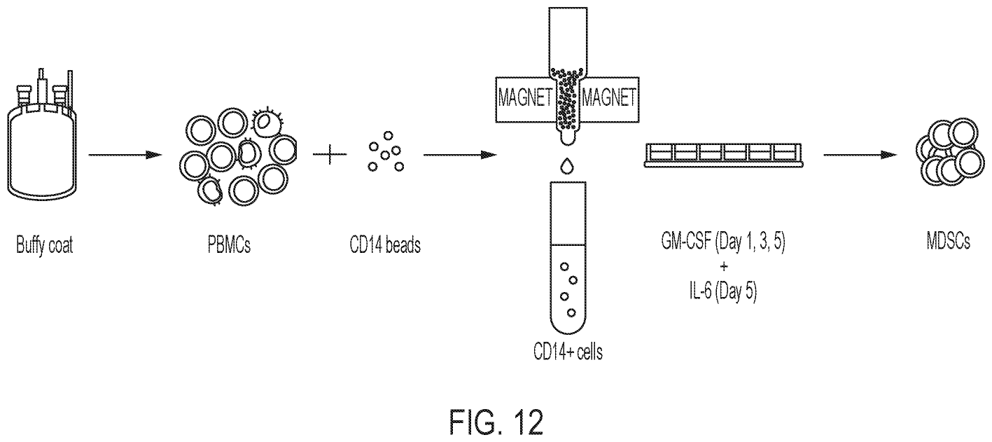

MDSCs were generated in vitro from healthy donor's buffy coat using protocols encompassed herein (FIG. 12). These MDSCs were characterized and validated as targets by confirming their expression of known phenotype markers for monocytic MDSCs including CD33+CD11b+ HLA-DR^{low} and CD14^{+ 27,28}. MDSCs also expressed high levels of the target, TRAIL-R2 on their surface (FIG. 2A). Furthermore, to assess the functionality of these MDSCs, T cell suppression assays were performed using autologous T cells isolated from the donor's PBMCs, and presence of MDSCs reduced T cell proliferation by 50±5% (FIGS. 2B-2C) and decreased T cell IFNg production by more than half compared to stimulated control T cells (342±134 pg/ml vs 1016±71.44 pg/ml, p=0.047) (FIG. 2D).

### Example 7

### MDSCS Lead to a Higher Tumor Burden, Increased Vascularization and Tumor Stroma Formation in TNBC Cell Derived Xenograft (CDX) Mouse Models

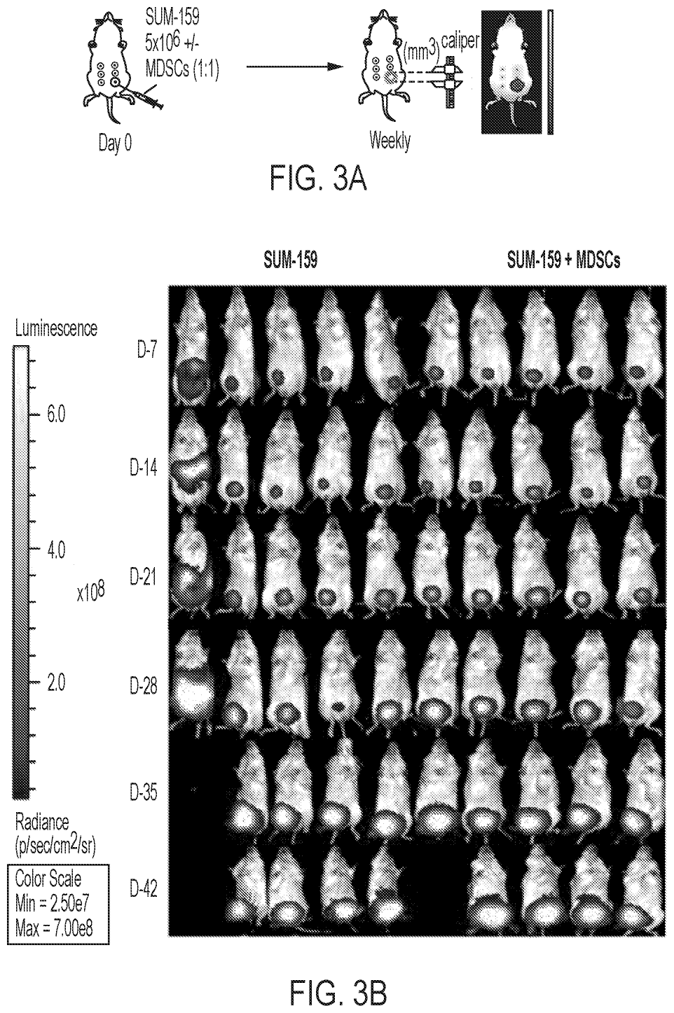

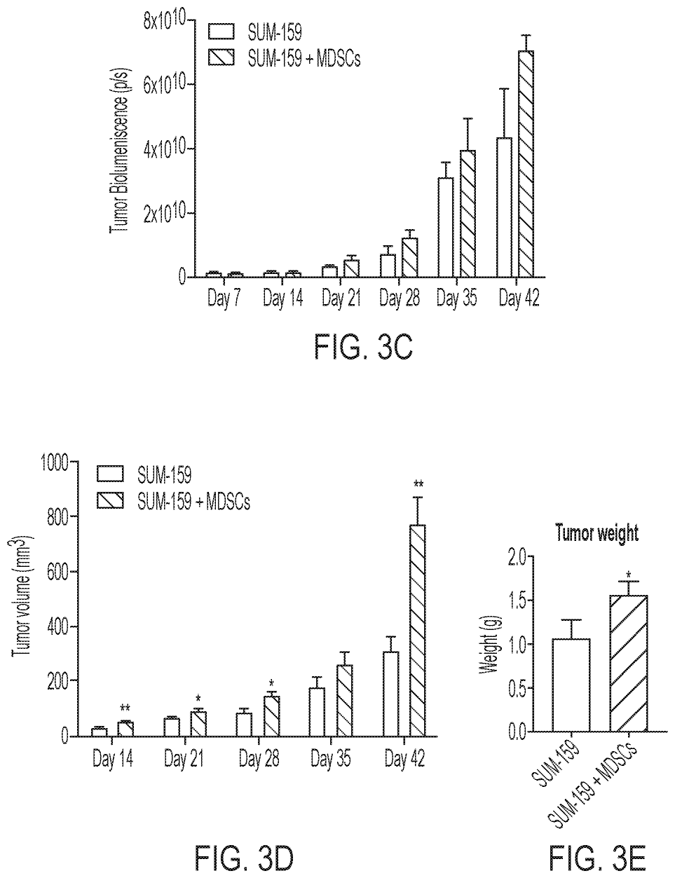

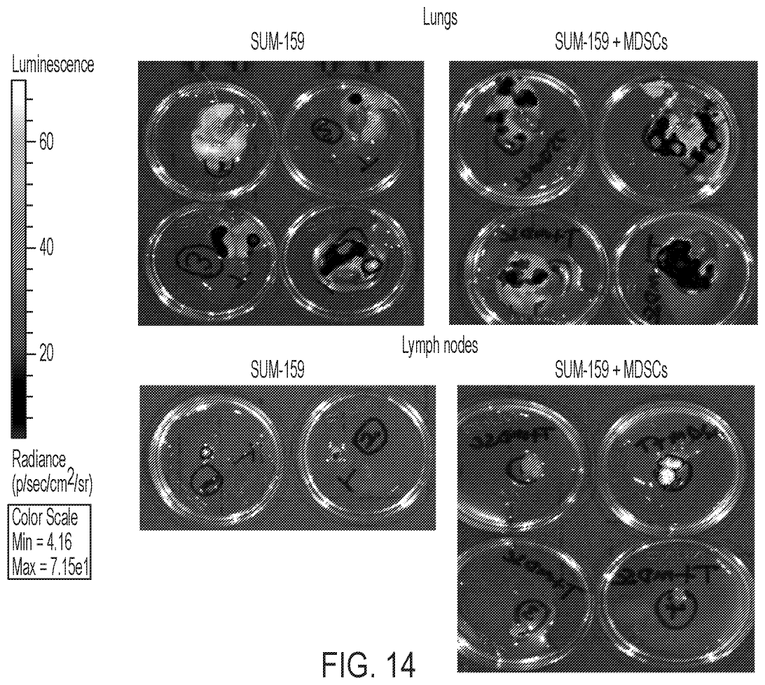

An in vivo mouse model was previously established to test the effects of the MUC1 CAR T cell therapy, by implanting TNBC cell line SUM-159 in the mammary fat pad of NSG mice. To test the effects of MDSCs in this model, 5×10⁶ tumor cells were co-injected with MDSCs at a 1:1 ratio. The SUM-159 cells were labelled with GFPff.Luc to allow tumor tracking in vivo by bioluminescence imaging. The tumor size was also assessed by weekly caliper measurement (FIG. 3A). As shown in FIGS. 3B-3C, there was a trend towards increase in the tumor burden when mice received tumor+MDSCs. These mice demonstrated a statistically significant increase in tumor volume (311.32±54.75 mm³ to 769.95±98.03 mm³, p=0.01) and weight (1.06±0.20 g to 1.56±0.15 g, p=0.03) compared to mice receiving tumor cells alone (FIGS. 3D-3E). Furthermore, the mice that received MDSCs showed a trend towards increased metastases (FIG. 14).

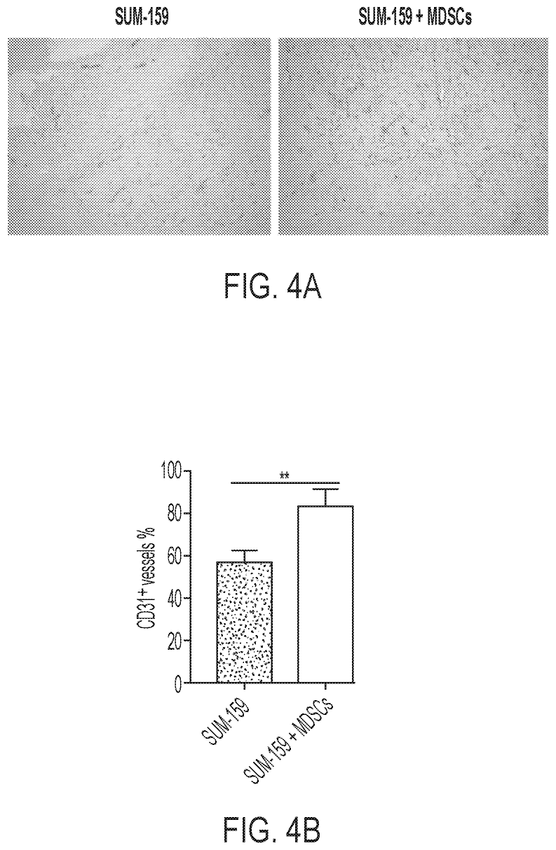

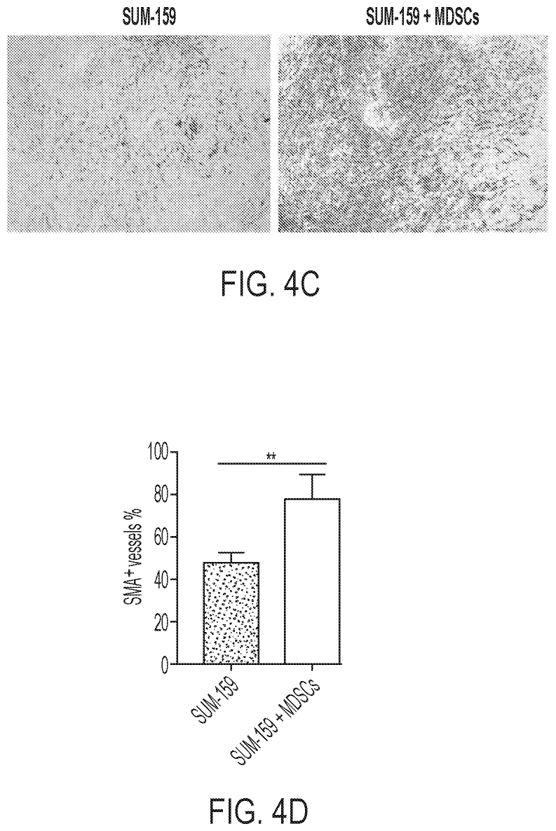

To further investigate the marked increase in tumor volume observed in presence of MDSCs, immunohistochemistry staining was used of the tumor tissues quantifying both CD31 expression as a surrogate marker of vascularization and fibroblast marker alpha-smooth muscle actin (a-SMA) expression as a measure of fibroblast accumulation. As shows in FIG. 4, tumors co-injected with MDSCs were associated with increased angiogenesis and greater accumulation of fibroblasts/stroma. Both of these mechanisms account for the increase in tumor volume and are known effects of MDSCs.

With all of these functional studies indicating that these in vitro generated MDSCs closely resemble MDSCs found in patients with breast cancer, these MDSCs were used to validate the novel TR2-41BB receptor.

### Example 8

### MDSCS Persist at the Tumor Site for 14 Days

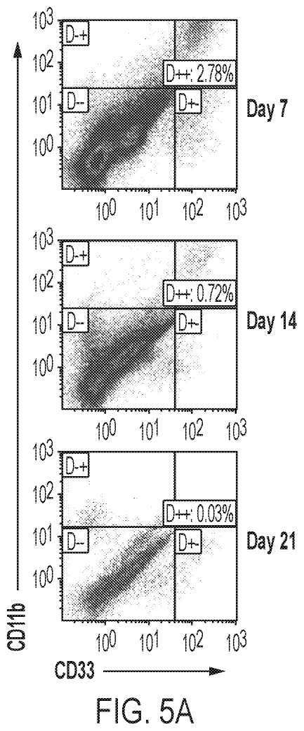

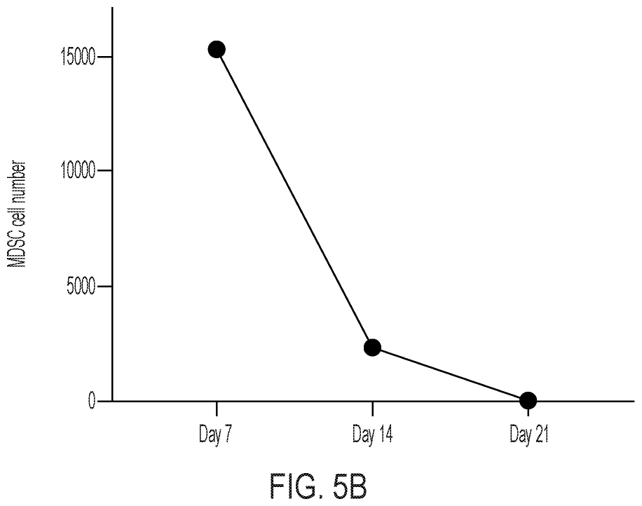

In order to determine the duration of MDSC persistence at the tumor site, NSG mice were co-injected with SUM-159 cells and MDSCs. The mice were then sacrificed at 7, 14 and 21 days and flow cytometry was performed to track their presence at the tumor site. To detect MDSCs, the tumors were stained with human anti-CD33 and anti-CD11b mAbs. As shown in FIG. 5A-5B, MDSCs were detected at day 7 after engraftment and by day 14 their number dropped by almost 7-fold. By day 21 they were no longer detected at the tumor site. Thus, MDSCs were detected at the tumor site for 14 days but not beyond day 21.

### Example 9

### Trail-R2.4-1Bb is Able to Rescue the Cytotoxic Activity of CAR.MUC1 in the Presence of MDSCS

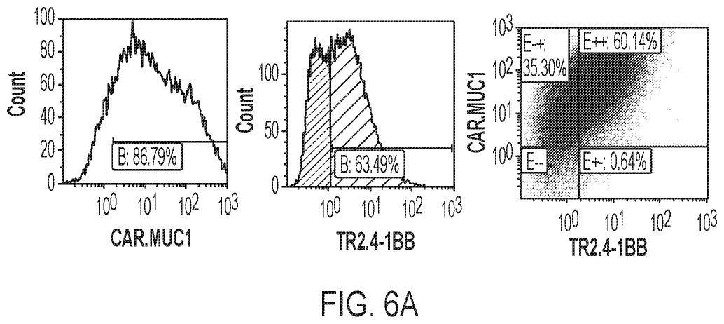

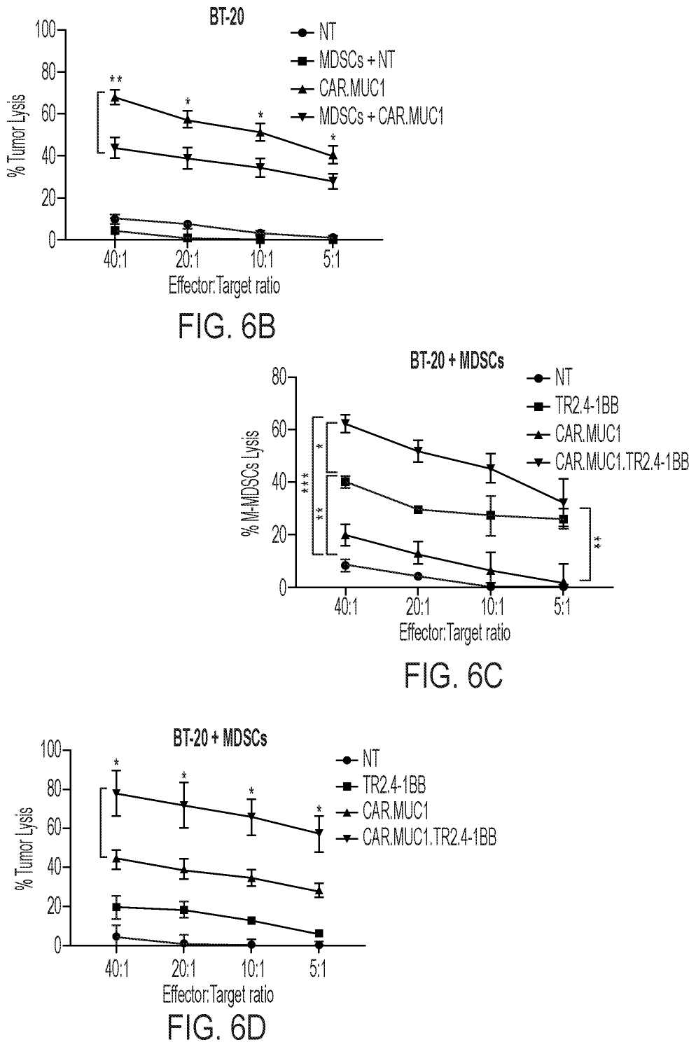

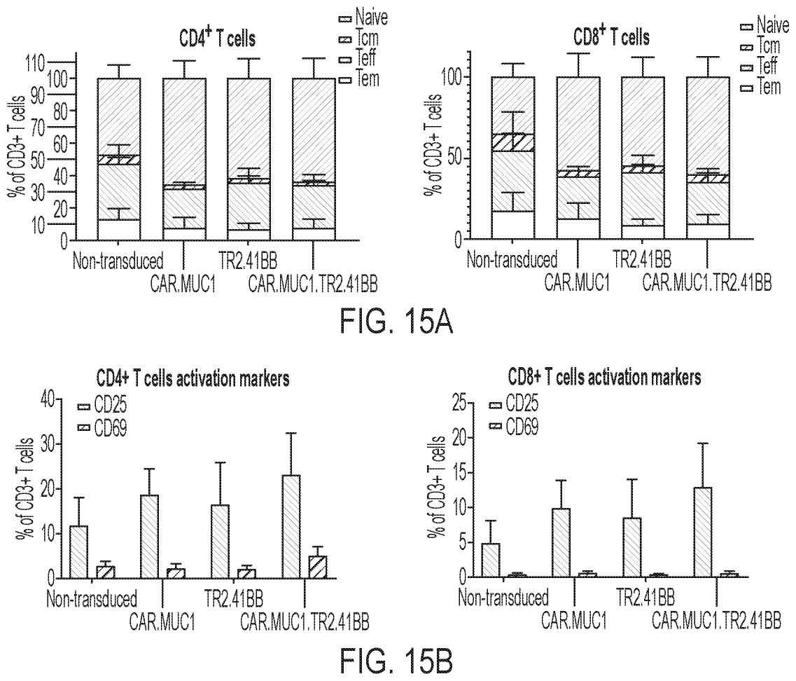

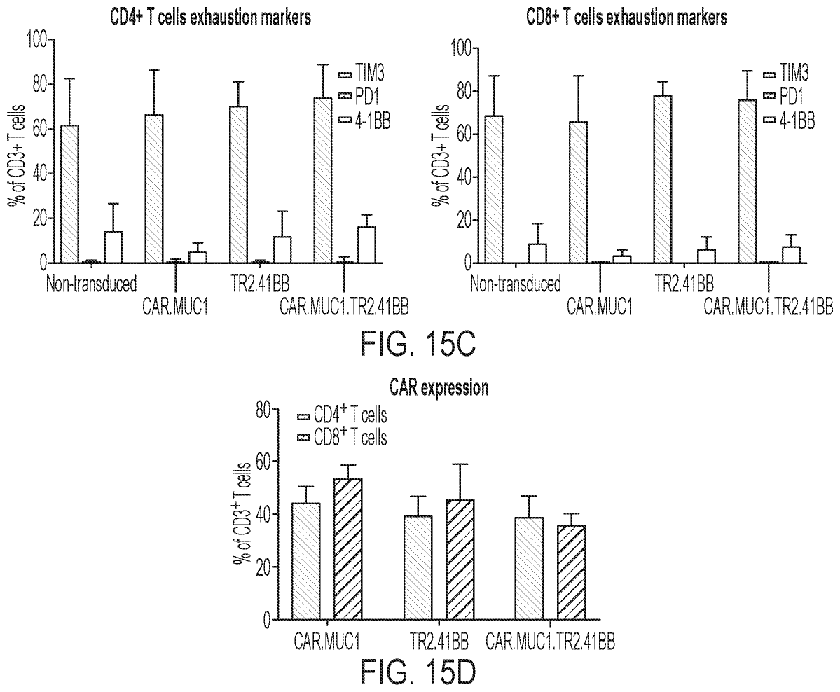

Activated T cells were co-transduced with both CAR.MUC1 and TRAIL-R2.4-1BB retroviral constructs with a good transduction efficiency, as measured by flow cytometry (87.3±4%, 80.8±2.7% and 74±5.5% for MUC1, TR2.4-1BB and MUC1.TR2.4-1BB CARTs, respectively) (FIG. 6A). The memory phenotype, activation and exhaustion status was assessed of the T cells by flow cytometry. There were no significant differences in the memory phenotype. Overall, all of the T cell products (CD4⁺ and CD8⁺ T cells) including NT T cells had similar percentages of naïve and central memory subsets, with T cell effector memory being higher (FIG. 15A). As shown in FIG. 15B-15C, no differences were noted as far as activation and exhaustion marker expression, increased levels of TIM3 and CD25 were observed across all T cell products and surface expression of CAR constructs was also comparable (FIG. 13D). To test whether the cytotoxic activity of CAR.MUC1 T cells is attenuated in the presence of MDSCs their cytolytic function was assessed in a 5 hr 51 chromium release assay using MUC1+BT-20 cells as targets either in the presence or absence of MDSCs. As shown in FIG. 6B, CAR.MUC1 T cells demonstrated almost 25% reduction in their cytotoxic potential in the presence of MDSCs (from 67.99±3.55% to 43.90±4.90%, p=0.003 for 40:1 effector to target ratio). Furthermore, to test MDSC apoptosis induced by the TRAIL-R2.4-1BB construct, labeled MDSCs were used as targets. The TRAIL-R2.4-1BB construct alone was able to induce almost 40% apoptosis of MDSCs at a 40:1 effector to target ratio, but this was not observed in the conditions with NT and CAR.MUC1 T cells. Additionally, CAR.MUC1.TRAIL-R2.4-1BB transduced T cells induced almost 60% MDSC lysis at the same ratio (FIG. 6C). Finally, it was assessed if introducing the TRAIL-R2.4-1BB construct together with CAR.MUC1 on T cells was able to rescue their cytotoxic potential in presence of MDSCs. As shown in FIG. 6D, addition of TRAIL-R2.4-1BB increased the cytolytic function of CAR.MUC1 T cells from 43.90±4.90% to 77.64±11.76%, p=0.014 at a 40:1 effector to target ratio. This nearly 30% increased cytotoxic potential was seen across all the effector to target ratios. This demonstrates the efficacy of the dual targeting approach in vitro.

### Example 10

### Expression of TR2.4-1Bb Enhances Expansion and Persistence of CAR.MUC1 T Cells and Augments their Anti-Tumor Activity In Vivo Despite Presence of MDSCS

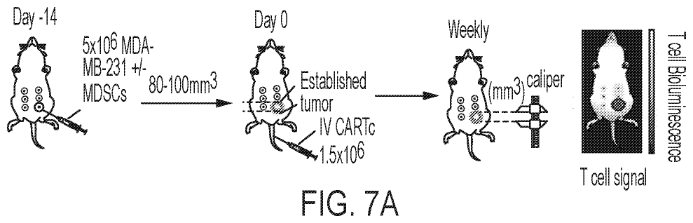

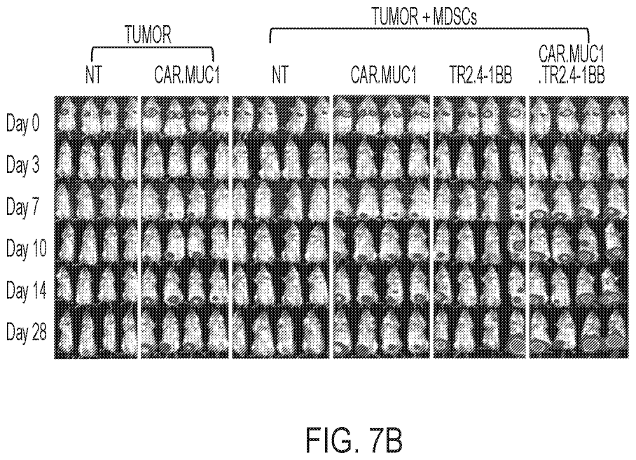

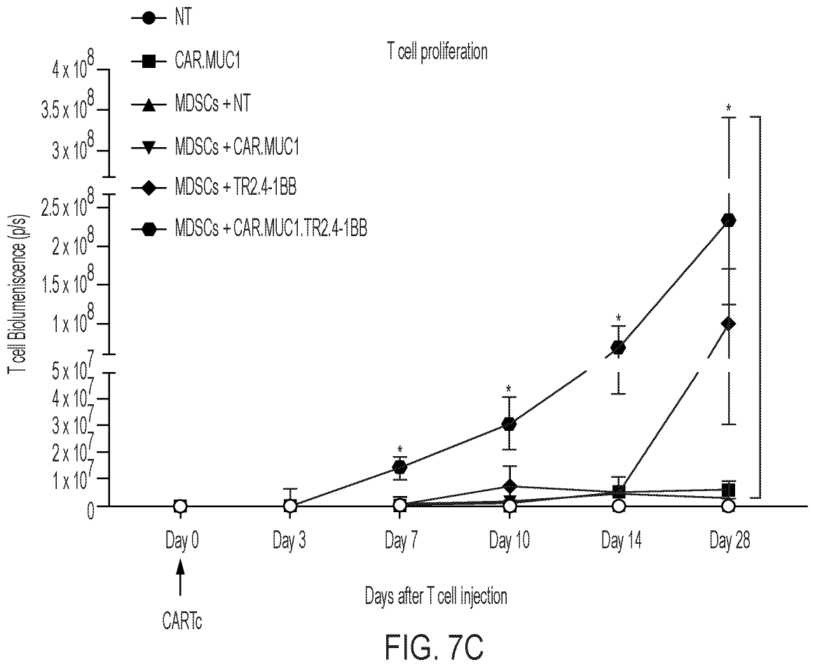

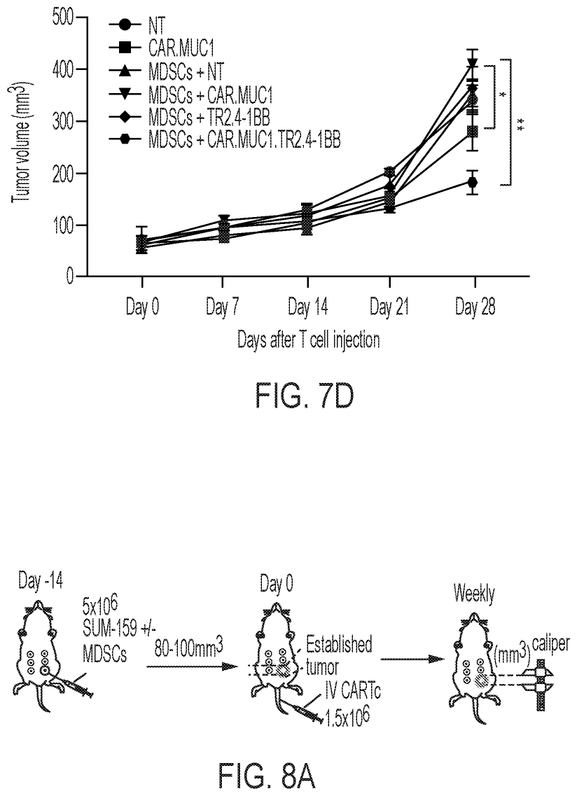

Finally, to test the efficacy of the CAR T cell therapy in vivo, NSG mice were engrafted (s.c. in the 4^{th} mammary fat pad) with 5×10⁶ MDA-MB-231 cells either in the presence or absence of 5×10⁶ MDSCs. Once the tumors had reached approximately 100 mm³ (˜2 weeks post-engraftment), mice were treated with 1.5×10⁶ GFP-FFLuc labelled NT, CAR.MUC1, TRAIL-R2.4-1BB or CAR.MUC1.TRAIL-R2.4-1BB T cells that were tracked in vivo by bioluminescence imaging (FIG. 7A). As shown in FIG. 7B-7C, all T cells in all conditions localized at the tumor site. However, CAR.MUC1.TRAIL-R2.4-1BB T cells demonstrated improved T cell proliferation and persistence at the tumor site in the presence of MDSCs compared to CAR.MUC1 or TRAIL-R2.4-1BB T cells alone (2.32×10⁸±1.07×10⁸p/s vs 3.06×10⁷±2.39×10⁷p/s, p=0.02 and 1.01×10⁸±7.02×10⁷p/s, p=0.05 for CAR.MUC1 and TR2.4-1BB respectively, on Day 28 after T cell injection). The detrimental effect of MDSCs in vivo was evident in this model, as the group receiving CAR.MUC1 T cells in the absence of MDSCs demonstrated better tumor control compared to the groups that had tumors with MDSCs treated with the same CAR.MUC1 T cells. This effect was rescued by introduction of the TR2.4-1BB receptor, whereby in the presence of MDSCs, only CAR.MUC1.TR2.4-1BB T cells were able to significantly decrease the rate of tumor growth (181.71±22.98 mm³) compared to either CAR.MUC1 or TR2.41BB alone (410.01±29.04 mm³, p=0.001 and 363.77±42.17 mm3, p=0.01 respectively, on Day 28 after T cell injection) (FIG. 7D).

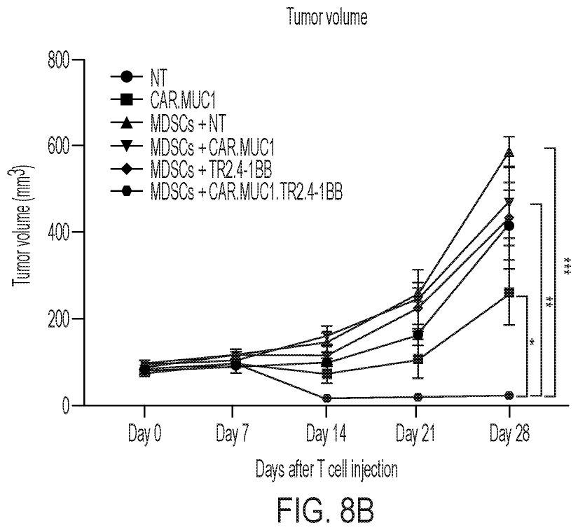

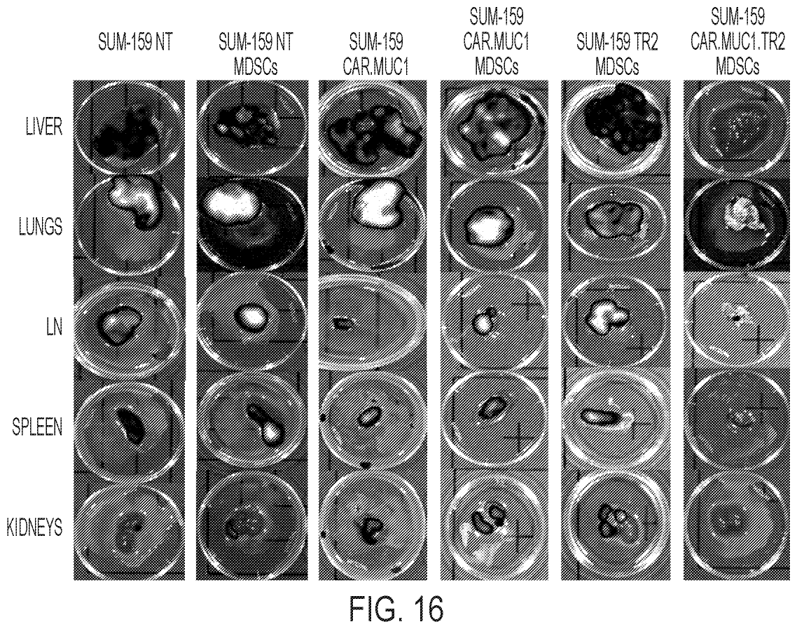

Additionally, the CAR T cell therapy was tested in a SUM-159 in vivo model; the tumor cells were labeled with GFP-FFLuc (FIG. 8A). Evidently, CAR.MUC1.TRAIL-R2.4-1BB T cells exhibited superior tumor cell killing and resulted in a significant reduction in tumor growth (24.54±8.55 mm³) compared to either CAR.MUC1 or TRAIL-R2.4-1BB (469.79.9±81.46 mm³, p=0.003 and 434.86±64.25 mm³, p=0.0004) respectively, on Day 28 after T cell injection) (FIG. 8B). Further, the metastatic burden was negligible in mice receiving CAR.MUC1.TRAIL-R2.4-1BB T cells compared to the other groups, thereby demonstrating their ability to eliminate the tumor and prevent further tumor cell dissemination (FIG. 16).

### Example 11

### Combined Expression of Trail-R2.4-1Bb and CAR.HER2 on T Cells Enhances Anti-Tumor Potential in a Her2+BC Model

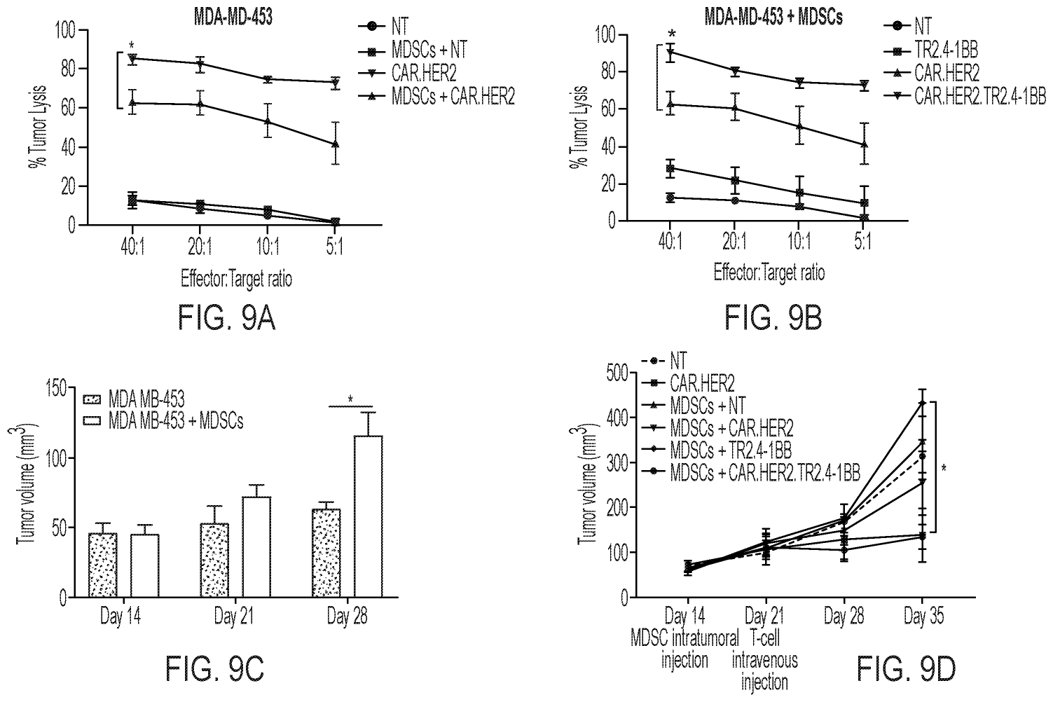

Additionally, to test the efficacy of TRAIL-R2.4-1BB construct in the context of another CAR, a HER2-specific CAR generated by Gottschalk et al. was obtained including the HER2-specific single chain variable fragment FRP5 into a SFG retroviral vector containing a CD28z signaling domain²⁶. Similar to CAR.MUC1 T cells, the in vitro cytotoxic potential of CAR.HER2 T cells against HER2+ MDA-MB-453 cells, measured by 51 chromium release assay, was attenuated in the presence of MDSCs (63.27±6.23% vs 84.87±2.68% for 40:1 effector to target ratio, p=0.05). (FIG. 9A). Expression of the TR2.4-1BB construct was able to restore the cytotoxic activity of HER2 CAR T cells in presence of MDSCs (from 63.40±6.13% to 90.40±4.97% for 40:1 effector to target ratio, p=0.041), (FIG. 9B). FIG. 9C shows increased tumor volume on administering MDSCs for the HER2+ CDX model. Since the MDA-MB-453 cell line is slow growing and MDSCs are undetectable by day 21, for this CDX model 5×10⁶ MDA-MB-453 cells were engrafted on day 0 and 5×10⁶ MDSCs were administered intratumorally on day 14. Once the tumors had reached approximately 100 mm³ (˜3 weeks post-engraftment) mice were treated with 1.5×10⁶ NT, CAR.HER2, TRAIL-R2.4-1BB or CAR.HER2.TRAIL-R2.4-1BB T cells. HER2.TR2.4-1BB CARTs led to reduced tumor volume (134.27±27.6 mm³) compared to CAR.HER2 (253.82±71.05 mm³) or TR2.4-1BB (432.24±29.71 mm³, p=0.01 on Day 14 after T cell injection) group receiving MDSCs (FIG. 9D). Thus, it was demonstrated using both the TNBC and HER2+BC models that CAR T cells co-expressing the novel TR2.4-1BB receptor have higher anti-tumor potential against BC tumors and infiltrating MDSCs, resulting in TME remodeling and improved T cell proliferation at the tumor site.

### Example 12

### Trail-R2.4-1Bb does not Induce Toxicity in Resting and Activated CD4⁺ and CD8⁺ T Cells

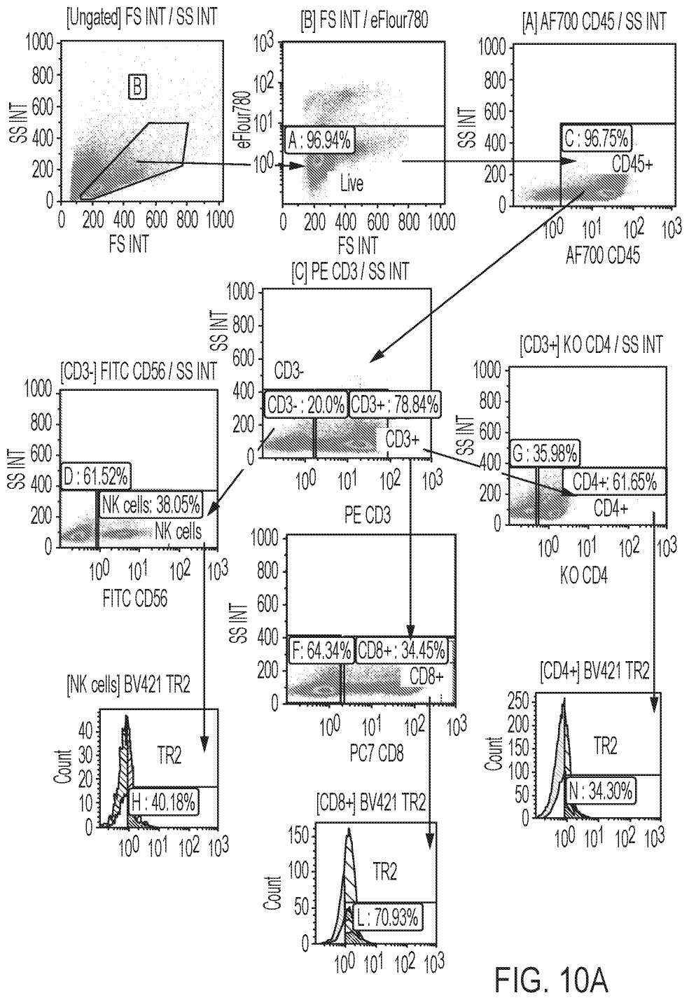

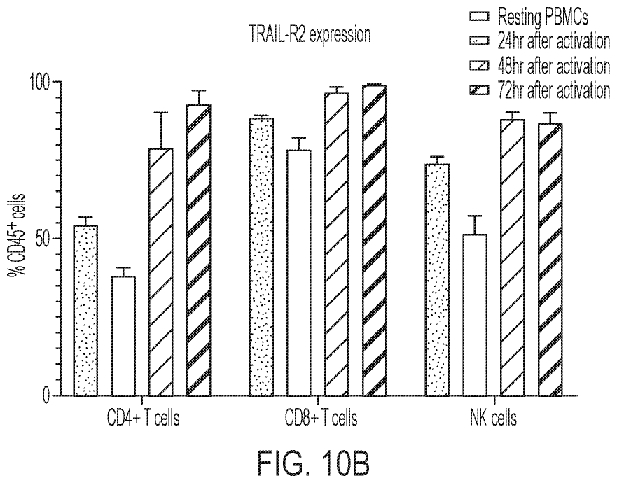

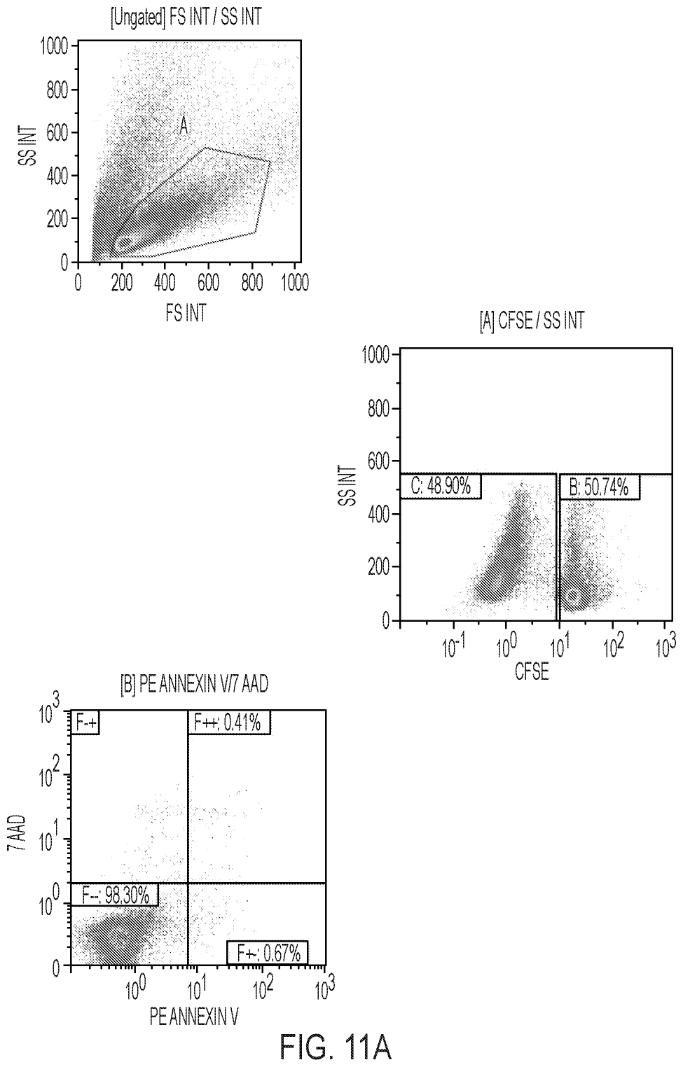

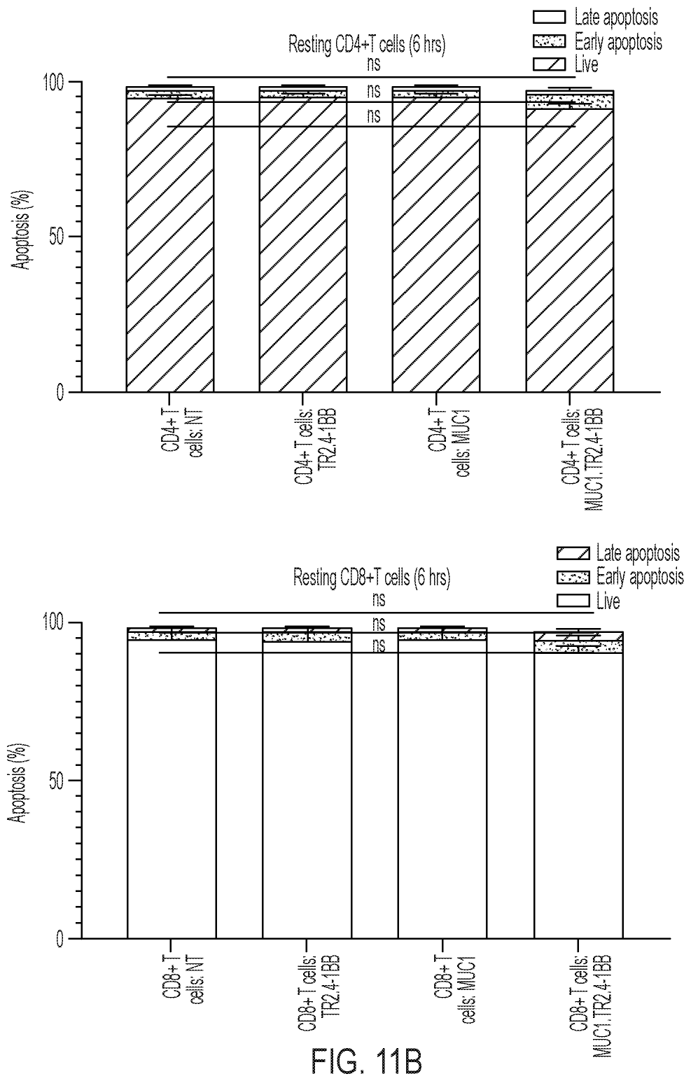

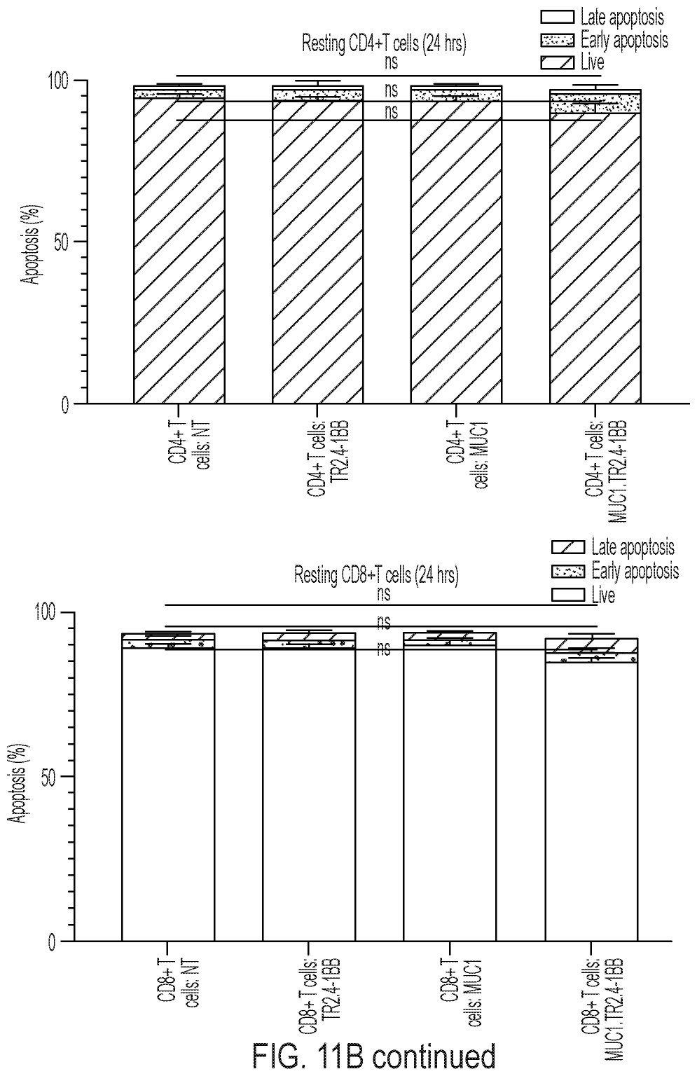

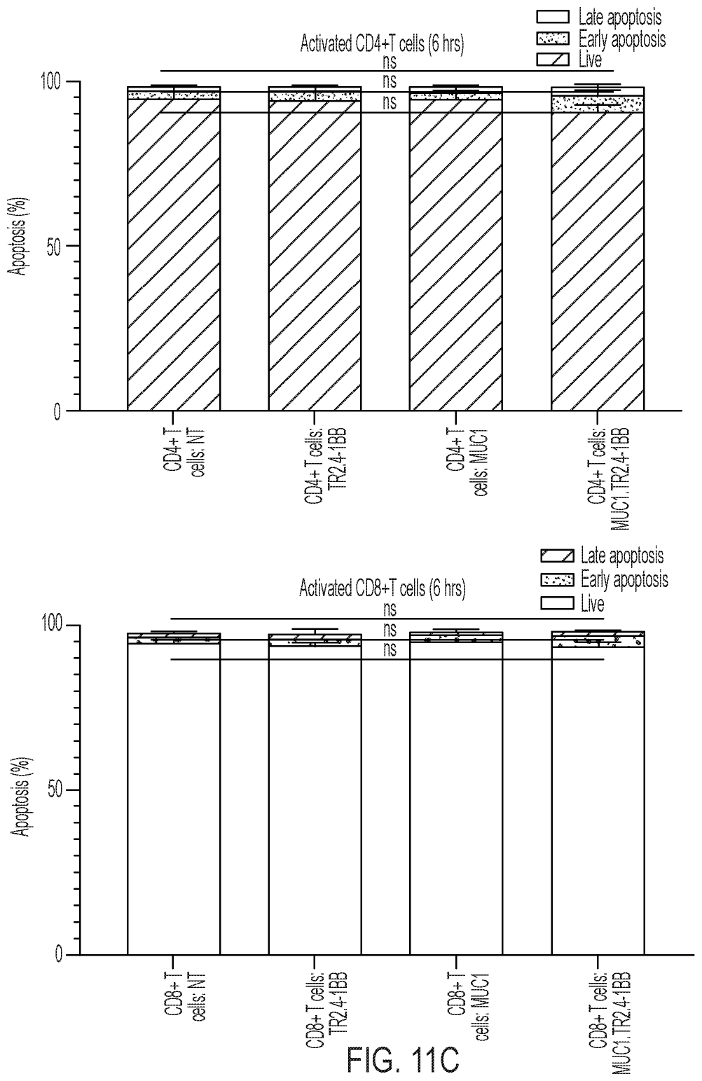

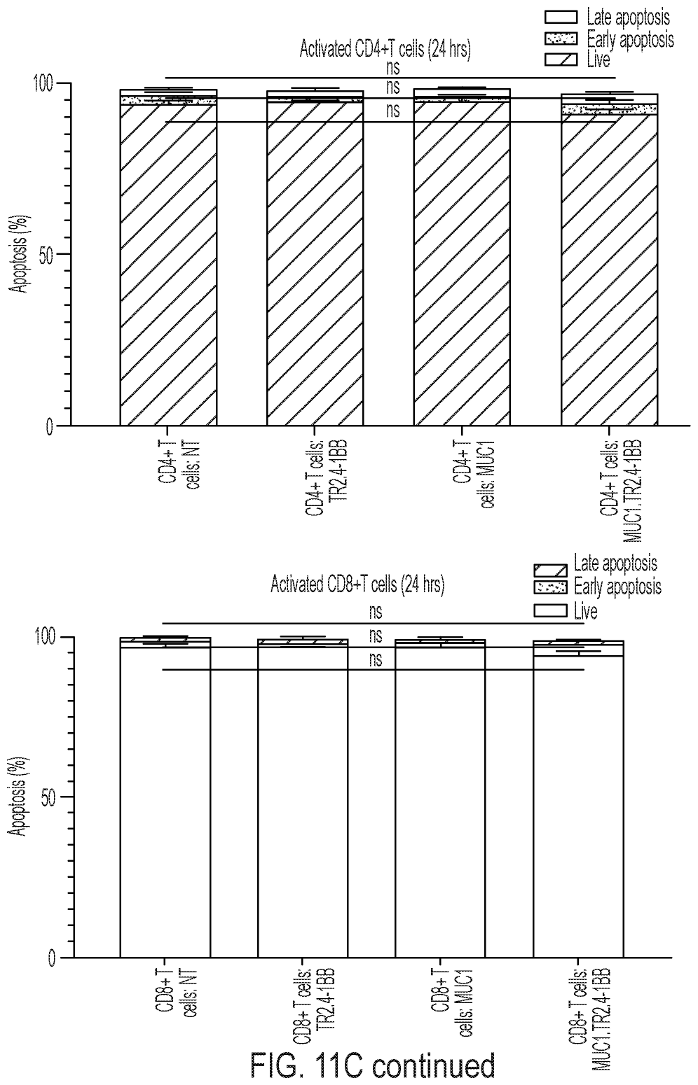

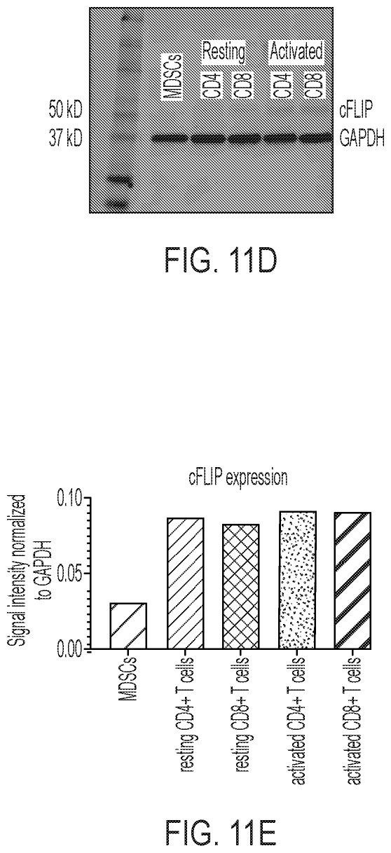

To test whether the TRAIL-R2.4-1BB construct is toxic to normal cells, the surface expression was assessed of TRAIL-R2 on resting and activated PBMCs at 24, 48 and 72 hrs by flow cytometry. Both resting and activated T cells showed high levels of surface TRAIL-R2 (FIGS. 10A-10B). Next, resting and activated cells were labelled with CFSE and they were co-cultured in the presence of T cells transduced with the transgenic constructs. The cells were then harvested at 6 hr and 24 hr, and the percentage of apoptotic cells was evaluated. There was no observed significant increase in the percentage of apoptosis in presence of the TR2.4-1BB construct compared to the NT or CAR.MUC1 groups (FIGS. 11A, 11B, and 11C). It was investigated whether the resistance to TRAIL-R2 mediated apoptosis in resting and activated T was due to increased expression of cellular FLICE-like inhibitory protein (cFLIP), a negative regulator of apoptosis^{29,30}. Using western blot, it was shown that both resting and activated CD4+ and CD8+ T cells expressed high levels of cFLIP compared to MDSCs (FIGS. 11D-11E). Thereby, this data indicates that TRAIL-R2.4-1BB does not induce toxicity in resting or activated T cells.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.
1. Siegel, R. L., Miller, K. D. & Jemal, A. Cancer statistics, 2019. CA. Cancer J. Clin. (2019) doi:10.3322/caac.21551.
2. Uscanga-Perales, G. I., Santuario-Facio, S. K. & Ortiz-López, R. Triple negative breast cancer: Deciphering the biology and heterogeneity. Med. Univ. (2016) doi:10.1016/j.rmu.2016.05.007.
3. Kumar, P. & Aggarwal, R. An overview of triple-negative breast cancer. Archives of Gynecology and Obstetrics (2016) doi:10.1007/s00404-015-3859-y.
4. Jackson, H. J., Rafiq, S. & Brentjens, R. J. Driving CAR T-cells forward. Nature Reviews Clinical Oncology (2016) doi:10.1038/nrclinonc.2016.36.
5. Gill, S., Maus, M. V. & Porter, D. L. Chimeric antigen receptor T cell therapy: 25 years in the making. Blood Reviews (2016) doi:10.1016/j.blre.2015.10.003.
6. Newick, K., O'Brien, S., Moon, E. & Albelda, S. M. CAR T Cell Therapy for Solid Tumors. Annu. Rev. Med. (2017) doi:10.1146/annurev-med-062315-120245.
7. Yu, F. et al. Deficiency of Kruppel-like factor KLF4 in mammary tumor cells inhibits tumor growth and pulmonary metastasis and is accompanied by compromised recruitment of myeloid-derived suppressor cells. Int. J. Cancer (2013) doi:10.1002/ijc.28302.
8. Sceneay, J., Parker, B. S., Smyth, M. J. & Möller, A. Hypoxia-driven immunosuppression contributes to the pre-metastatic niche. Oncoimmunology (2013) doi:10.4161/onci.22355.
9. Zhang, Y. et al. A novel role of hematopoietic CCL5 in promoting triple-negative mammary tumor progression by regulating generation of myeloid-derived suppressor cells. Cell Res. (2013) doi:10.1038/cr.2012.178.
10. Cao, Y. et al. BMP4 inhibits breast cancer metastasis by blocking myeloid-derived suppressor cell activity. Cancer Res. (2014) doi:10.1158/0008-5472.CAN-13-3171.
11. Morales, J. K., Kmieciak, M., Knutson, K. L., Bear, H. D. & Manjili, M. H. GM-CSF is one of the main breast tumor-derived soluble factors involved in the differentiation of CD11b-Gr1-bone marrow progenitor cells into myeloid-derived suppressor cells. Breast Cancer Res. Treat. (2010) doi:10.1007/s10549-009-0622-8.
12. Oh, K. et al. A mutual activation loop between breast cancer cells and myeloid-derived suppressor cells facilitates spontaneous metastasis through IL-6 trans-signaling in a murine model. Breast Cancer Res. (2013) doi:10.1186/bcr3473.
13. Diaz-Montero, C. M. et al. Increased circulating myeloid-derived suppressor cells correlate with clinical cancer stage, metastatic tumor burden, and doxorubicin-cyclophosphamide chemotherapy. Cancer Immunol. Immunother. (2009) doi:10.1007/s00262-008-0523-4.
14. Almand, B. et al. Clinical significance of defective dendritic cell differentiation in cancer. Clin. Cancer Res. (2000).
15. Alshetaiwi, H. et al. Defining the emergence of myeloid-derived suppressor cells in breast cancer using single-cell transcriptomics. Sci. Immunol. (2020) doi:10.1126/sciimmunol.aay6017.
16. Markowitz, J., Wesolowski, R., Papenfuss, T., Brooks, T. R. & Carson, W. E. Myeloid-derived suppressor cells in breast cancer. Breast Cancer Research and Treatment (2013) doi:10.1007/s10549-013-2618-7.
17. Morgan, M. A. & Schambach, A. Engineering CAR-T Cells for Improved Function Against Solid Tumors. Frontiers in immunology (2018) doi:10.3389/fimmu.2018.02493.
18. Condamine, T. et al. ER stress regulates myeloid-derived suppressor cell fate through TRAIL-R-mediated apoptosis. J. Clin. Invest. (2014) doi:10.1172/JCI74056.
19. Dominguez, G. A. et al. Selective targeting of myeloid-derived suppressor cells in cancer patients using DS-8273a, an agonistic TRAIL-R2 antibody. Clin. Cancer Res. (2017) doi:10.1158/1078-0432.CCR-16-1784.
20. Bajgain, P. et al. CAR T cell therapy for breast cancer: Harnessing the tumor milieu to drive T cell activation. J. Immunother. Cancer (2018) doi:10.1186/s40425-018-0347-5.
21. Tarp, M. A. et al. Identification of a novel cancer-specific immunodominant glycopeptide epitope in the MUC1 tandem repeat. Glycobiology (2007) doi:10.1093/glycob/cwl061.
22. Müller, S. et al. High density O-glycosylation on tandem repeat peptide from secretory MUC1 of T47D breast cancer cells. J. Biol. Chem. (1999) doi:10.1074/jbc.274.26.18165.
23. Kim, M. J. et al. Novel antibodies targeting MUC1-C showed anti-metastasis and growth-inhibitory effects on human breast cancer cells. Int. J. Mol. Sci. (2020) doi:10.3390/ijms21093258.
24. Dominguez, G. A. et al. Selective targeting of myeloid-derived suppressor cells in cancer patients using DS-8273a, an agonistic TRAIL-R2 antibody. Clin. Cancer Res. (2017) doi:10.1158/1078-0432.CCR-16-1784.
25. Bajgain, P. et al. CAR T cell therapy for breast cancer: Harnessing the tumor milieu to drive T cell activation. J. Immunother. Cancer (2018) doi:10.1186/s40425-018-0347-5.
26. Ahmed, N. et al. Immunotherapy for osteosarcoma: Genetic modification of T cells overcomes low levels of tumor antigen expression. Mol. Ther. (2009) doi:10.1038/mt.2009.133.
27. Bronte, V. et al. Recommendations for myeloid-derived suppressor cell nomenclature and characterization standards. Nature Communications (2016) doi:10.1038/ncomms12150.
28. Casacuberta-Serra, S. et al. Myeloid-derived suppressor cells can be efficiently generated from human hematopoietic progenitors and peripheral blood monocytes. Immunol. Cell Biol. (2017) doi:10.1038/icb.2017.4.
29. Clarke, P. & Tyler, K. L. Down-regulation of cFLIP following reovirus infection sensitizes human ovarian cancer cells to TRAIL-induced apoptosis. Apoptosis (2007) doi:10.1007/s10495-006-0528-4.
30. He, M.-X. & He, Y.-W. c-FLIP Protects T Lymphocytes from Apoptosis in the Intrinsic Pathway. J. Immunol. (2015) doi:10.4049/jimmunol.1400469.

What is claimed is:

1. A polynucleotide comprising an expression construct that encodes a chimeric protein, wherein said chimeric protein comprises (1) an extracellular region comprising a TNF-related apoptosis-inducing ligand Receptor 2 (TRAIL-R2)-binding agent; and (2) an intracellular region that is not from TRAIL-R2.

2. The polynucleotide of claim 1, wherein the TRAIL-R2-binding agent is an antibody or functional fragment thereof.

3. The polynucleotide of claim 2, wherein the antibody is an scFv of a monoclonal antibody specific for TRAIL-R2.

4. The polynucleotide of claim 1, wherein the TRAIL-R2-binding agent is a ligand of TRAIL-R2.

5. The polynucleotide of claim 4, wherein the ligand of TRAIL-R2 is TRAIL.

6. The polynucleotide of any one of claims 1-5, wherein the intracellular domain is further defined as comprising one or more costimulatory domains and optionally CD3zeta.

7. The polynucleotide of claim 6, wherein the one or more costimulatory domains is from CD28, CD137 (4-1BB), CD134 (OX40), DAP10, DAP12, NKG2D, CD40L, CD27, CD2, CD5, ICAM-1, LFA-1 (CD11a/CD18), Lck, TNFR-I, TNFR-II, Fas, CD30, CD40, 2B4, DNAM, CS1, CD48, NKp30, NKp44, NKp46, NKp80, members of the TNFR superfamily, or a combination thereof.

8. The polynucleotide of claim 7, wherein the costimulatory domain is from 4-1BB.

9. The polynucleotide of any one of claims 1-8, wherein the transmembrane domain of the chimeric protein is from CD28, the alpha chain of the T-cell receptor, beta chain of the T-cell receptor, zeta chain of the T-cell receptor, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, DAP10, or DAP12.

10. The polynucleotide of any one of claims 1-8, wherein the TRAIL-R2-binding agent is an scFv of a monoclonal antibody specific for TRAIL-R2 and the intracellular region comprises the costimulatory domain from 4-1BB.

11. The polynucleotide of any one of claims 1-10, wherein the polynucleotide is a vector.

12. The polynucleotide of claim 11, wherein the vector is a viral vector or a non-viral vector.

13. The polynucleotide of claim 12, wherein the viral vector is an adenoviral vector, lentiviral vector, retroviral vector, or adeno-associated viral vector.

14. The polynucleotide of claim 12, wherein the non-viral vector comprises a plasmid, transposon, nanoparticle, liposome, lipid, metal, or a combination thereof.

15. An isolated cell, comprising the polynucleotide of any one of claims 1-14.

16. The cell of claim 15, wherein the cell is a eukaryotic or prokaryotic cell.

17. The cell of claim 16, wherein the eukaryotic cell is a human cell.

18. The cell of claim 17, wherein the human cell is an immune cell or stem cell.

19. The cell of claim 18, wherein the immune cell is a T cell, NK cell, NKT cell, MSC, monocyte, macrophage, B cell, or a mixture thereof.

20. The cell of claim 19, wherein the T cell is an αβ T cell or a γδ T cell.

21. The cell of claim 17, wherein the stem cell is a mesenchymal stem cell, hematopoietic stem cell, induced pluripotent stem cell, or a mixture or derivative thereof.

22. The cell of any one of claims 15-21, wherein the cell comprises a vector that encodes a therapeutic protein.

23. The cell of claim 22, wherein the therapeutic protein is a native or an engineered antigen receptor.

24. The cell of claim 23, wherein the engineered antigen receptor is a chimeric antigen receptor (CAR) or a transgenic T cell receptor.

25. The cell of claim 24, wherein the vector that encodes the CAR and the vector that encodes the chimeric protein are different.

26. The cell of claim 24, wherein the vector that encodes the CAR and the vector that encodes the chimeric protein are the same vector.

27. The cell of any one of claims 24-26, wherein the CAR targets a cancer antigen, neo-antigen, or endogenous antigen expressed by the tumor or other cells at the tumor site.

28. The cell of claim 27, wherein the cancer antigen is selected from the group consisting of Zona pellucida sperm-binding protein 4 (ZP4), Melanoma-associated antigen D4 (MAGE-D4), CD19, EBNA, CD123, HER2, CA-125, TRAIL/DR4, TRAIL-R2, CD20, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, -2, -8, GAGE-3, -4, -5, -6, -7B, NA88-A, MC1R, mda-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WTi), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, STATE, hypoxia inducible factors (e.g., HIF-1 and HIF-2), Nuclear Factor-Kappa B (NF-B), Notch receptors (e.g., Notch1-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, and LRRN1.

29. The cell of any one of claims 24-28, wherein the CAR comprises one or more costimulatory domains selected from the group consisting of 4-1BB, CD27, CD28, DAP12, NKG2D, OX-40 (CD134), DAP10, CD40L, 2B4, DNAM, CS1, CD48, NKp30, NKp44, NKp46, NKp80, and a combination thereof.

30. The cell of any one of claims 24-29, wherein the CAR comprises a transmembrane domain selected from the group consisting of CD28, the alpha chain of the T-cell receptor, beta chain of the T-cell receptor, zeta chain of the T-cell receptor, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, DAP10, and DAP12.

31. The cell of any one of claims 27-30, wherein the neo-antigen is a PIK3CA mutation, ESR1 mutation, TP53 mutation, AKT mutation, or a combination thereof.

32. The cell of any one of claims 27-31, wherein an engineered or native TCR targets the neo-antigen.

33. A method of inhibiting TRAIL-R2-expressing cells in a tumor microenvironment of an individual, comprising the step of administering to the individual a therapeutically effective amount of the cells of any one of claims 15-32.

34. The method of claim 33, wherein the cells are administered more than once.

35. The method of claim 34, wherein the duration of time between administrations is 1, 2, 3, 4, 5, 6, 7, or more days.

36. The method of any one of claims 33-35, wherein the cells are administered in the range of about 10³ to 10¹⁰ cells per dose.

37. The method of any one of claims 33-36, wherein the administering is intravenously, intradermally, transdermally, intrathecally, intraarterially, intraperitoneally, intranasally, intravaginally, intrarectally, topically, intramuscularly, subcutaneously, mucosally, orally, topically, locally, inhalation, injection, infusion, continuous infusion, localized perfusion bathing target cells directly, via a catheter, via a lavage, in cremes, in lipid compositions, or a combination thereof.

38. The method of claim 37, wherein the administering is by infusion.

39. The method of any one of claims 33-38, wherein the individual is administered an additional cancer therapy.

40. The method of claim 39, wherein the additional cancer therapy is surgery, radiation, drug therapy, immunotherapy, hormone therapy, or a combination thereof.

41. A method of treating cancer in an individual, comprising the step of administering to the individual a therapeutically effective amount of the cells of any one of claims 15-32.

42. The method of claim 41, wherein the cells are administered more than once.

43. The method of claim 42, wherein the duration of time between administrations is 1, 2, 3, 4, 5, 6, 7, or more days.

44. The method of any one of claims 41-43, wherein the cells are administered in the range of about 10³ to 10¹⁰ cells per dose.

45. The method of any one of claims 41-44, wherein the administering is intravenously, intradermally, transdermally, intrathecally, intraarterially, intraperitoneally, intranasally, intravaginally, intrarectally, topically, intramuscularly, subcutaneously, mucosally, orally, topically, locally, inhalation, injection, infusion, continuous infusion, localized perfusion bathing target cells directly, via a catheter, via a lavage, in cremes, in lipid compositions, or a combination thereof.

46. The method of claim 45, wherein the administering is by infusion.

47. The method of any one of claims 41-46, wherein the individual is administered an additional cancer therapy.

48. The method of claim 47, wherein the additional cancer therapy is surgery, radiation, drug therapy, immunotherapy, hormone therapy, or a combination thereof.

49. A method of enhancing a cellular therapy for cancer in an individual, comprising the step of modifying cells of the cellular therapy to express the polynucleotide of any one of claims 1-14.

50. The method of claim 49, wherein the cells of the cellular therapy express one or more chimeric antigen receptors, transgenic receptors, or non-transgenic receptors.
